(19) United States
(12) Patent Application Publication
(10) Pub. No.: US 2022/0273842 A1
(43) Pub. Date: Sep. 1, 2022

(54) A TISSUE-ADHESIVE MATRIX AND USES THEREOF

(71) Applicant: NURAMI MEDICAL LTD., Haifa (IL)

(72) Inventors: Nora NSEIR MANASSA, Haifa (IL); Amir BAHAR, Qiryat Tivon (IL); Rodaina OMAR, Kfar Kana (IL)

(21) Appl. No.: 17/629,387

(22) PCT Filed: Jul. 22, 2020
(86) PCT No.: PCT/IL2020/050815

§ 371 Date: Jan. 23, 2022

Related U.S. Application Data
(60) Provisional application No. 62/876,952, filed on Jul. 22, 2019.

(51) Int. Cl. A61L 24/04; A61L 24/00

(52) U.S. Cl. CPC A61L 24/043; A61L 24/0015; A61L 24/0036

(57) ABSTRACT

Compositions, comprising a first polymer, a second branched polymer, and a third polymer having reactivity to the second polymer, wherein any of the second and the third polymer comprises a tissue-adhesive group, and wherein the second polymer and the third polymer are at least partially crosslinked are disclosed. Matrices, comprising the composition of the invention and optionally an additional polymeric layer are disclosed. Processes for manufacturing the compositions and uses thereof as for bioadhesion and/or repairing damaged tissues are also disclosed.

### CROSS-REFERENCE TO RELATED APPLICATIONS

This application claims the benefit of priority of U.S. Provisional Patent Application No. 62/876,952 filed Jul. 22, 2019, the contents of which are incorporated herein by reference in their entirety.

### FIELD OF THE INVENTION

The present invention, in some embodiments thereof, relates to a tissue-adhesive matrix, preparation and use thereof.

### BACKGROUND OF THE INVENTION

Leakage of liquid or air from or into a damaged tissue is a potentially life-threatening condition which may occur as a result of a wide variety of circumstances, including surgery and traumatic injury.

Soft tissues are particularly prone to damage. Additionally, these tissues sometimes create various compartments that hold liquid or air (e.g., lungs, blood vessels, dura matter, urinary bladder, etc.), and when damaged, their impairment can extend to other areas as well. Furthermore, because of the mechanical nature of these tissues, the attachment of a matrix with sutures or staples can cause damage in and of itself, e.g., to prevent proper sealing, to increase the probability of bacterial infection or to reduce the rate of recovery or recuperation. Examples of such soft tissues include dura mater, brain tissue, retina, skin tissue, hepatic tissue, pancreatic tissue, connective tissue, muscle tissue, cardiac tissue, vascular tissue, renal or urogenital tissue, pulmonary tissue, gonadal tissue, hematopoietic tissue, digestive tract tissue (such as colon or stomach) and fat tissue.

Adhesion to tissue without suturing may be provided by an adhesive (e.g., applied on a surface) which promotes cell growth attachment (e.g., growth factors, extracellular matrix proteins, and/or other proteins). Polymerizable compositions have been used in various adhesives, for example as dental materials or as adhesives for holding reconstructive elements in place. Currently available tissue adhesives have some inherent shortcomings, which limit their use in the clinic. Cyanoacrylate-based glues, for example, adhere very strongly to tissues, but are associated with severe inflammatory response and poor elasticity. Hydrogels, on the other hand, are considered safe, but lack the adhesion strength required for holding the tissue together. As a result, the use of cyanoacrylates is limited to external surfaces, while hydrogels such as fibrin glue serve as sealant-they seal the wound rather than hold it together. Therefore, new adhesion strategies that offer a viable alternative to suture and staples are much needed.

### SUMMARY OF THE INVENTION

In one aspect of the invention, there is provided a composition comprising:

a first polymer;

a second branched polymer;

a third polymer having reactivity to the second polymer and is at least partially crosslinked to the second branched polymer;

wherein any one of the second and the third polymer comprises a tissue-adhesive group.

In one embodiment, an average molecular weight of the first polymer ranges from 10 KDa to 900 KDa.

In one embodiment, the first polymer is selected from the group comprising: a polyester, a polyanhydride, a polyacetal, a polyorthoester, a polyurethane, a polycarbonate, a polyphosphazene, a polyphosphoester, a polyether, a silicone, a polyamide, a polysulfone, a polyether ether ketone (PEEK), poly(ethylene glycol), polytetrafluoroethylene, polyethylene, a polysaccharide or any combination or a copolymer thereof.

In one embodiment, the third polymer is branched.

In one embodiment, the third polymer comprises a nucleophilic group.

In one embodiment, crosslinked is by reacting the tissue-adhesive group and the nucleophilic group.

In one embodiment, the branched polymer is selected from the group consisting of: a star polymer, a dendrimer, and a hyperbranched polymer or any combination thereof.

In one embodiment, the branched polymer comprises three to ten arms.

In one embodiment, the tissue-adhesive group is selected from the group consisting of: an activated ester (e.g. thio-ester, a peu-oroalkyl ester, a pento-orophenol ester, a N-hydroxysuccinimide ester), an acyl halide, a chloroformate, an anhydride, an aldehyde, an epoxide, an isocyanate, an isothiocyanate, a maleimide, a carbonate, a sulfonyl chloride, a haloacetamide, an acyl azide, an imidoester, a carbodiimide, a vinyl sulfone, ortho-pyridyl-disulfide, or any combination thereof.

In one embodiment, the tissue-adhesive group is covalently bound to an arm of the second polymer.

In one embodiment, the second polymer, the third polymer, or both are selected from the group consisting of: polyethers, polyesters, polydioxanones, polyphosphoesters, polyurethanes, and polyamides or any combination or a co-polymer thereof.

In one embodiment, the second polymer, the third polymer, or both comprise polyethyleneglycol; and wherein the first polymer is selected from the group consisting of: polylactic acid, poly(L-lactic acid), poly(D-lactic acid), polyglycolic acid, poly(L-glycolic acid), poly(D-glycolic acid), nylon and polycaprolactone or any combination or a co-polymer thereof.

In one embodiment, an average molecular weight of the second polymer, and of the third polymer ranges from 500 Da to 100000 Da.

In one embodiment, a weight ratio of the third polymer to the second branched polymer ranges from 1:1 to 1:10.

In one embodiment, a weight ratio of the first polymer to the second branched polymer ranges from 1:1 to 20:1.

In one embodiment, the first polymer, and at least one of the second branched polymer and the third polymer are blended together, so as to form a blended polymeric fiber.

In one embodiment, the blended polymeric fiber is biodegradable.

In one embodiment, the blended polymeric fiber is characterized by an average fiber diameter of 0.5 to 10 um.

In one embodiment, the blended polymeric fiber is characterized by a melting point of 50 to 150° C.

In another aspect, there is provided a matrix, comprising a tissue adhesive layer, wherein the tissue adhesive layer comprises a blended polymeric fiber of the invention.

In one embodiment, the matrix further comprises an additional layer of polymeric fibers.

In one embodiment, the additional layer enhances a stability of the tissue-adhesive layer.

In one embodiment, the tissue-adhesive layer is characterized by a pore size of 0.5 to 100 um.

In one embodiment, the tissue-adhesive layer is characterized by a tensile strength of at least 0.05 MPa.

In one embodiment, the tissue-adhesive layer is characterized by an adhesion strength of 1 to 10 N, wherein the adhesion strength is measured according to a shear test.

In one embodiment, the tissue-adhesive layer is characterized by a porosity of at least 60%.

In one embodiment, the tissue-adhesive layer is characterized by a thickness of 0.5 to 250 um.

In one embodiment, the tissue-adhesive layer is characterized by a water-permeability of less than 1 ml per hour per cm2 upon exposure to an aqueous liquid at a pressure of 40 mmHg.

In one embodiment, the matrix further comprises a pharmaceutically active ingredient.

In one embodiment, the matrix is for use in use in promoting (i) bioadhesion of at least one biological tissue; (ii) blood coagulation.

In one embodiment, the matrix is for use in repairing and/or substituting a biological tissue.

In another aspect, there is a kit comprising (i) a blended polymeric fiber comprises a first polymer and a second polymer; and (ii) a composition comprising a third polymer having reactivity to the second polymer; wherein the second polymer and the third polymer comprise respectively the second branched polymer or the third polymer of the invention.

In one embodiment, the first polymer is selected from the group comprising: a polyester, a polyanhydride, a polyacetal, a polyorthoester, a polyurethane, a polycarbonate, a polyphosphazene, a polyphosphoester, a polyether, a silicone, a polyamide, a polysulfone, a polyether ether ketone (PEEK), poly(ethylene glycol), polytetrafluoroethylene, polyethylene, a polysaccharide or a combination or a co-polymer thereof.

In one embodiment, the second polymer, the third polymer, or both comprise polyethyleneglycol; and wherein the first polymer is selected from the group consisting of: polylactic acid, poly(L-lactic acid), poly(D-lactic acid), polyglycolic acid, poly(L-glycolic acid), poly(D-glycolic acid), nylon and polycaprolactone or any combination or a co-polymer thereof.

In one embodiment, the blended polymeric fiber in contact with the additional component results in a tissue-adhesive layer.

In one embodiment, the weight ratio of the third polymer to the second polymer ranges from 1:1 to 1:20.

In one embodiment, the weight ratio of the first polymer to the second polymer ranges from 1:1 to 20:1.

In another aspect, there is a process for manufacturing the composition of the invention or the blended polymeric fiber of the kit of the invention, comprising: (i) mixing a first polymer and at least one of the second polymer and the third polymer with a solvent, thereby obtaining a solution; and (ii) providing the solution into an electrospinning apparatus.

In one embodiment, the process is for manufacturing a layer of polymeric fibers.

### BRIEF DESCRIPTION OF THE DRAWINGS

FIG. 1 presents a general illustration of the peel test.

FIG. 2 presents a general illustration of the shear test.

FIG. 3 presents SEM images of the electro-spun samples. FIG. 3A: Control 1.2 FIG. 3B: Composition 1.2.

FIG. 4 presents a bar graph depicting the fiber diameter of the exemplary electrospun samples. Controls are assigned as cont.

FIG. 5 presents a bar graph depicting the pore size of the exemplary electrospun samples. Controls are assigned as cont.

FIG. 6 presents a bar graph depicting the tensile strength of the exemplary electro-spun samples. Controls are assigned as cont.

FIGS. 7A-7B present bar graphs depicting the adhesion strength, as determined by the peel test. FIG. 7A presents average peel force exhibited by exemplary samples and controls. FIG. 7B presents maximum force exhibited by exemplary samples and controls. Controls are assigned as cont.

FIG. 8 presents a bar graph depicting the adhesion strength of the exemplary samples and controls, as determined by the shear test.

FIG. 9 presents a bar graph depicting burst pressure strength of the exemplary samples and controls.

### DETAILED DESCRIPTION OF THE INVENTION

In one aspect, the present invention, relates to a composition comprising a first polymer, a second polymer comprising a tissue adhesive group and a third polymer, wherein the second polymer and the third polymer are at least partially crosslinked. In some embodiments, the present invention relates to a composition, being in a form of a blended polymeric fiber.

In another aspect, the present invention is related to a tissue adhesive matrix comprising the blended polymeric fiber of the invention. Further, the present invention provides a method of manufacturing the matrix and uses thereof such as for tissue adhesion.

The present invention is based, in part, on the surprising finding that a tissue-adhesive matrix comprising a cross-linked polymer exhibited an enhanced adhesive strength, as compared to matrices comprising a linear tissue-adhesive polymer.

### Composition

In some embodiments, there is provided a composition comprising: a first polymer; a second branched polymer; a third polymer having reactivity to the second polymer and is at least partially crosslinked to the second branched polymer; wherein any one of the second and the third polymer comprises a tissue-adhesive group.

In some embodiments, the first polymer is a carrier polymer. In some embodiments, the first polymer provides a structural support to the composition comprising the same.

As used herein, the term “structural support” is related to physical properties of the composition (e.g. a blended polymeric fiber), such as elasticity. Additionally, the first polymer may be selected so as to enable a polymeric fiber formation by any one of the methods described herein below (e.g. by electrospinning). In some embodiments, the first polymer provides a stability to the polymeric fiber.

As used herein, the terms “elasticity” and “elastic” refer to a tendency of a material to return to its original shape after being deformed by stress, for example, a tensile stress and/or shear stress, at an indicated temperature or at a temperature of 37° C. (in contexts wherein no temperature is indicated). The elasticity may be expressed by tensile properties.

The elongation at failure is determined as the maximal strain (elongation) which can occur (upon application of tensile stress equal to the tensile strength) before failure of the tested material occurs (e.g., as rupture or necking).

In some embodiments, the first polymer is a synthetic polymer. In some embodiments, the first polymer is selected form the group comprising: a polyester, a polyanhydride, a polyacetal, a polyorthoester, a polyurethane, a polycarbonate, a polyphosphazene, a polyphosphoester, a polyether, a silicone, a polyamide, a polysulfone, a polyether ether ketone (PEEK), a poly(ethylene glycol), polytetrafluoroethylene, polyethylene, and a mixture or a copolymer thereof.

In some embodiments, the first polymer is biodegradable. In some embodiments, the first polymer is at least partially biodegradable and/or bioderodible. In some embodiments, the first polymer is substantially biodegradable and/or bioderodible, wherein substantially is as described herein.

In some embodiments, the first polymer is a copolymer, comprising poly(lactic acid). In some embodiments, the first polymer is poly(lactic acid). In some embodiments, the first polymer comprises a polyester. In some embodiments, the first polymer comprises at least one biodegradable polyester.

Non-limiting examples of polyesters include but are not limited to polyglycolide, polylactic acid, polycaprolactone (PCL), polyhydroxyalkanoate, polyhydroxybutyrate, polyethylene adipate, polybutylene succinate, poly(3-hydroxybutyrate-co-3-hydroxyvalerate), polyethylene terephthalate (PET), polybutylene terephthalate, polyethylene naphthalate (PEN), including any copolymer or any combination thereof.

In some embodiments, the first polymer comprises a poly(alpha-hydroxy)carboxilic acid. In some embodiments, the first polymer is a co-polymer, comprising a first polymeric segment comprising a poly(alpha-hydroxy)carboxilic acid; and a second polymeric segment comprising a polyester.

In some embodiments, the first polymer is a co-polymer comprising a plurality of polyesters. In some embodiments, the first polymer is a co-polymer comprising a polyester selected from polylactide, polyglycolide, polycaprolactone (PCL), and optionally comprising a polyamide (e.g. nylon). In some embodiments, the first polymer comprises polylactide-co-polycaprolactone (PLA-co-PCL). In some embodiments, the first polymer comprises polyglycolide-co-polycaprolactone. In some embodiments, the first polymer comprises polyglycolide-co-polycaprolactone (PLGA-co-PCL). In some embodiments, the first polymer comprises poly(L-glycolide)-co-polycaprolactone. In some embodiments, the first polymer comprises poly(D-glycolide)-co-polycaprolactone. In some embodiments, the first polymer comprises poly(L-lactide)-co-poly(ε-caprolactone) (PLLA-PCL), poly(D,L-lactide)-co-poly(ε-caprolactone), poly(D-lactide)-co-poly(ε-caprolactone) or any combination thereof. In some embodiments, the first polymer is a biological polymer. In some embodiments, the biological polymer is selected form the group comprising: a polysaccharide, a polypeptide, a polynucleic acid and a mixture or a copolymer thereof. In some embodiments, the biological polymer comprises a chemical modification (e.g. crosslinking, acetylation, methylation, hydrolysis).

In some embodiments, the biological polymer is a polysaccharide. Non-limiting examples of polysaccharides include but are not limited to: cellulose acetate, gum arabic, gum ghatti, dextran, pullulan, amylopectin, and hyaluronic acid. In some embodiments, the biological polymer induces blood coagulation. In some embodiments, the biological polymer comprises collagen, oxidized cellulose or both.

In some embodiments, the first polymer is characterized by an average molecular weight ranging from 10000 Da to 900,000 Da, from 10000 Da to 100,000 Da, from 10,000 Da to 50,000 Da, from 50,000 Da to 100,000 Da, from 100,000 Da to 200,000 Da, from 200,000 Da to 300,000 Da, from 300,000 Da to 400,000 Da, from 400,000 Da to 500,000 Da, from 500,000 Da to 600,000 Da, from 600,000 Da to 900,000 Da, including any range or value therebetween. In some embodiments, the first polymer is characterized by an average molecular weight ranging from 50 to 70 kDa, from 70 to 100 kDa, from 100 to 150 kDa, from 150 to 200 kDa, from 200 to 250 kDa, from 250 to 300 kDa, including any range or value therebetween.

In some embodiments, any of the first polymer, the second branched polymer, and the third polymer substantially comprises a single homopolymer or a single copolymer. In some embodiments, any of the first polymer, the second branched polymer, and the third polymer is substantially devoid of a particulate matter (e.g. a nano-particle, a micro-particle organic or inorganic). In some embodiments, any of the first polymer, the second branched polymer, and the third polymer is substantially devoid of a non-biodegradable polymer and/or a non-biodegradable polymeric segment. In some embodiments, the composition of the invention consists essentially of the first polymer, the second branched polymer, and the third polymer. In some embodiments, the first polymer, the second branched polymer, and the third polymer compose at least 80%, at least 85%, at least 90%, at least 95%, at least 97%, at least 98%, at least 99%, at least 99.5%, at least 99.9%, by weight of the dry content of the composition of the invention. In some embodiments, the first polymer, the second branched polymer, and the third polymer compose at least 80%, at least 85%, at least 90%, at least 95%, at least 97%, at least 98%, at least 99%, at least 99.5%, at least 99.9%, by weight of the blended polymeric fiber of the invention. In some embodiments, any one of the first polymer, the second branched polymer, and the third polymer is substantially devoid of acrylate modified PEG-PLLA co-polymer. In some embodiments, the tissue-adhesive group is substantially devoid of acrylate. In some embodiments, the tissue-adhesive group is substantially devoid of vinyl sulfone. In some embodiments, the composition of the invention is substantially devoid of a polyamino acid (e.g. a peptide).

In some embodiments, the first polymer is a high-molecular weight polymer. In some embodiments, an average molecular weight of the first polymer is greater by at least 100%, at least 200%, at least 300%, at least 400%, at least 500%, at least 600%, at least 700%, at least 800%, at least 900%, at least 1000%, than an average molecular weight of any one of the second and the third polymer, including any range or value therebetween.

In some embodiments, the first polymer is characterized by a tensile strength and elongation at failure, which is greater than a tensile strength and elongation at failure of any one of the second and the third polymer, wherein greater is as described hereinabove.

In some embodiments, the w/w ratio of the first polymer to the total weight of the composition is at least 10%, at least 15%, at least 20%, at least 25%, at least 30%, at least 35%, at least 40%, at least 45%, including any range or value therebetween.

In some embodiments, the w/w ratio of the first polymer to the total weight of the composition is at most 20%, at most 25%, at most 30%, at most 35%, at most 40%, at most 45%, at most 50% including any range or value therebetween. In some embodiments, the w/w ratio of the first polymer to the total weight of the composition is at most 50%.

In some embodiments, the w/w ratio of the first polymer from the total weight of the composition is between 10 and 60%, between 10 and 20%, between 20 and 30%, between 30 and 40%, between 40 and 50%, between 50 and 60%, including any range or value therebetween.

In some embodiments, the composition (e.g. in a form of a fiber) having the w/w content of the first polymer being between 20 and 60%, between 30 and 50%, between 40 and 50%, or at most 50% is characterized by a sufficient adhesion strength (e.g. above 1.1N), wherein the adhesion strength is as described herein.

In some embodiments, the composition comprises a second polymer. In some embodiments, the second polymer is a branched polymer. In some embodiments, a branched polymer is selected from the group consisting of: a star polymer, a dendrimer, and a hyperbranched polymer or any combination thereof. In some embodiments, the terms “second polymer” and “second branched polymer” are used herein interchangeably.

In some embodiments, a branched polymer (e.g. the second branched polymer and/or the third polymer) comprises a branched core. In some embodiments, the branched core is covalently linked to at least three arms.

Non-limiting examples of branched cores include but are not limited to: pentaerythritol, dipentaerythritol, tripentaerythritol, calyx[8]arene, or any combination thereof.

In some embodiments, the branched polymer (e.g. the second polymer and/or the third polymer) comprises the branched core covalently linked to three or more arms, wherein each one of the arms has the same chemical composition.

In some embodiments, the branched polymer (e.g. the second polymer and/or the third polymer) comprises the branched core covalently linked to three or more arms, wherein at least some of the arms have a different chemical composition.

As used herein, the term “chemical composition” describes a composition matter of any one of the segments (e.g. a chemical structure and average number of monomers in a polymeric segment).

In some embodiments, the branched polymer (e.g. the second polymer and/or the third polymer) has between 3 and 10, between 3 and 5, between 5 and 7, between 7 and 8, between 8 and 10 arms, including any range or value therebetween. In some embodiments the branched polymer (e.g. the second polymer and/or the third polymer) has 3-8 arms. In some embodiments, the branched polymer (e.g. the second polymer and/or the third polymer) has 4-8 arms. In some embodiments, the branched polymer (e.g. the second polymer and/or the third polymer) has 3-6 arms. In some embodiments, the branched polymer (e.g. the second polymer and/or the third polymer) has 4 arms. In some embodiments, the branched polymer (e.g. the second polymer and/or the third polymer) has 8 arms. In some embodiments, the branched polymer (e.g. the second branched polymer and/or the third polymer of the invention) comprises 8 arms. In some embodiments, at least 80%, at least 85%, at least 90%, at least 92%, at least 95%, at least 97%, at least 99% by total weight of the branched polymers (e.g. the second branched polymer and/or the third polymer of the invention) comprises 8 arms.

In some embodiments, any one of the arms of the branched polymer (e.g. the second polymer and/or the third polymer) independently comprises a polymeric segment. In some embodiments, any one of the arms of the branched polymer (e.g. the second polymer and/or the third polymer) independently comprises at least one polymeric segment, and at least one tissue-binding group.

As used herein, the term “polymeric segment” refers to a polymeric structure of any length. In the art of polymer technology, a long polymeric structure is often referred to as a block, whereas a short polymeric structure is often referred to as a segment. Both these conventional meanings are understood to be comprised in the term “segment” as used herein.

In some embodiments, the polymeric segment of the branched polymer (e.g. the second polymer and/or the third polymer) is a copolymer comprising a plurality of polymeric subunits. In some embodiments, the copolymer is selected from the group consisting of: block-, alternating-, periodic-, and random-copolymers.

In some embodiments, the polymeric segment of the branched polymer (e.g. the second polymer and/or the third polymer) is a homopolymer.

In some embodiments, the polymeric segment of the branched polymer (e.g. the second polymer and/or the third polymer) comprises at least one biodegradable subunit. In some embodiments, the polymeric segment comprises at least one biocompatible subunit. In some embodiments, the polymeric segment comprises at least one biocompatible and biodegradable subunit. In some embodiments, the polymeric segment comprises at least one biodegradable subunit and at least one non-biodegradable subunit. In some embodiments, the polymeric segment is fully biodegradable. In some embodiments, the polymeric segment is fully biocompatible. In some embodiments, the polymeric segment is biodegradable and biocompatible.

As used herein, the term “biocompatible”, is intended to describe materials that, are non-toxic to cells in vitro and upon administration in vivo, do not induce undesirable long-term effects.

As used herein, the term “biodegradable”, is intended to describe materials comprising covalent bonds that are degraded in vivo, wherein the degradation of the covalent bond occurs via hydrolysis. The hydrolysis can involve a direct reaction with an aqueous medium or can be catalyzed chemically or enzymatically. “Aqueous medium” refers to water, aqueous solutions, physiological media or biological fluids (e.g., body fluids), and other pharmaceutically acceptable media. Suitable hydrolysable covalent bonds are selected from the group containing: esters, amides, urethanes, carbamates, carbonates, ethers, azo linkages, anhydrides, thioesters, and combinations thereof.

Non-limiting examples of biodegradable polymers include but are not limited to: polyethers (e.g. polyethylenglycole (PEG)), polyglycolides, polyesters (e.g. poly-l-lactide (PLLA), polycaprolactones, polyhydroxybutyrate, polyhydroxyvalerate), polydioxanones, polyurethanes, polyphosphoesters, polyurethanes, and polyamides (e.g. polyamino acids) including any co-polymer or any combination thereof.

In some embodiments, the polymeric segment of the second polymer and/or of the third polymer comprises PEG. In some embodiments, the second polymer, the third polymer or both comprise a polyester. In some embodiments, the second polymer, the third polymer or both comprise a polyether. In some embodiments, the second polymer, the third polymer or both comprise PEG.

In some embodiments, the polymeric segment of the second polymer has reactivity to the third polymer. In some embodiments, the polymeric segment of the second polymer comprises a reactive group. In some embodiments, the reactive group of the second polymer has reactivity to a reactive group of the third polymer. In some embodiments, the reactive group of the third polymer has reactivity to a reactive group of the second polymer. In some embodiments, a reactive group is capable of covalent bond formation with a third polymer. In some embodiments, the reactive group of the second polymer is an electrophile. In some embodiments, a reactive group of the second polymer is a tissue-adhesive group. In some embodiments, the second polymer comprises a tissue-adhesive group (e.g. an electrophile or an electrophilic tissue-adhesive group), wherein the tissue-adhesive group has a reactivity to the third polymer. In some embodiments, the tissue-adhesive group of the second polymer has a reactivity to a reactive group (e.g. a nucleophilic group as described herein) of the third polymer. In some embodiments, the second polymer and the third polymer are capable of forming a covalent bond via a reaction of the tissue-adhesive group of the second polymer with the reactive group of the third polymer. In some embodiments, the second polymer comprises a tissue-adhesive group covalently linked to the polymeric segment thereof. In some embodiments, the first polymer is substantially devoid of a reactive group, wherein the reactive group is as described herein. In some embodiments, the first polymer is substantially inert (e.g. unreactive). In some embodiments, the first polymer is substantially inert (e.g. unreactive) to any of the second polymer and to the third polymer.

In some embodiments, the polymeric segment of the second polymer comprises one type of tissue-adhesive groups or more types of tissue-adhesive groups.

The term “tissue-adhesive group” encompasses any chemical group or functionality that may interact with a biological surface (e.g. a tissue), resulting in a covalent or non-covalent bond formation. Biological surfaces, such as tissues generally consist of cells, comprising on their surface protein molecules, which commonly contain thiol and primary amine moieties. Many functional groups such as activated ester, may covalently bind to the biological surface by reacting with thiols or primary amines, which are located on the cell surface. In addition to forming covalent bonds, the tissue-adhesive groups may form non-covalent bonds with the biological surface. The term “non-covalent bonds” encompasses any one of ligand-receptor interactions, hydrogen bonding, dipole-dipole interactions and van der Waals bonds or any combination thereof. The use of tissue-adhesive groups in terms of present invention, provides bioadhesive properties to the polymeric material.

As used herein, the term “biological surface” refer to any surface comprising cells and/or biological molecules (e.g. proteins, polysaccharides, lipids, nucleic acids). Non-limiting examples of “biological surface” include, but are not limited to tissue surface, synthetic graft surface, and organ surface.

Non-limiting examples of tissue-adhesive groups, which form non-covalent bonds with the biological surface, include, but are not limited to amides, carboxylates and peptides (e.g. RGD).

Non-limiting examples of tissue-adhesive groups which form covalent bonds with the biological surface, include, but are not limited to: an activated ester (e.g. thio-ester, a peu-oroalkyl ester, a N-hydroxysuccinimide ester), a carboxylic acid, an acyl halide, a chloroformate, an anhydride, an aldehyde, an epoxide, an isocyanate, an isothiocyanate, a maleimide, a carbonate, a sulfonyl chloride, a haloacetamide, an acyl azide, an imidoester, a carbodiimide, a vinyl sulfone, ortho-pyridyl-disulfide, or any combination thereof.

In some embodiments, the tissue-adhesive group is an activated ester.

In some embodiments, the tissue-adhesive group is N-hydroxysuccinimide (NHS) ester. The mechanism by which an NHS-functionalized polymer reacts with an amine-containing material, such as a tissue protein, is illustrated below.

In some embodiments, the tissue-adhesive group is covalently linked to a terminal group of the polymeric segment.

In some embodiments, the tissue-adhesive group is covalently linked to a side chain of the polymeric segment.

In some embodiments, a plurality of tissue-adhesive groups provide bioadhesive properties to the second polymer.

In some embodiments, the polymeric segment of the second polymer comprises tissue-adhesive monomers, which form covalent and/or non-covalent bonds with the biological surface, resulting in bioadhesion.

In some embodiments, the composition comprises a third polymer.

In some embodiments, a third polymer is biodegradable.

In some embodiments, a third polymer is selected from the group consisting of: polyethers (e.g. polyethylenglycole (PEG)), polyglycolides, polyesters (e.g. poly-l-lactide (PLLA), polycaprolactones, polyhydroxybutyrate, polyhydroxyvalerate), polydioxanones, polyurethanes, polyphosphoesters, polyurethanes, and polyamides (e.g. polyamino acids) or any combination thereof.

In some embodiments, the second polymer, the third polymer, or both comprise PEG.

In some embodiments, an average molecular weight of the third polymer and of the second polymer ranges from 500 to 100000 Da, from 500 to 5000 Da, from 1000 to 3000 Da, from 1500 to 2500 Da, from 5000 to 10000 Da, from 10000 to 15000 Da, from 15000 to 18000 Da, from 18000 to 20000 Da, from 20000 to 22000 Da, from 22000 to 25000 Da, from 25000 to 30000 Da, from 30000 to 40000 Da, from 40000 to 60000 Da, from 60000 to 80000 Da, from 80000 to 100000 Da, or any range therebetween.

In some embodiments, an average molecular weight of any one of the second polymer and of the third polymer is between 1000 and 50.000 Da. In some embodiments, an average molecular weight of at least one of the second polymer and of the third polymer is between 10.000 and 50.000 Da, between 10.000 and 20.000 Da, between 20.000 and 30.000 Da, between 30.000 and 40.000 Da, between 40.000 and 50.000 Da, including any range or value therebetween. In some embodiments, the composition (e.g. a fiber) comprises a first polymer and at least one of the second polymer and of the third polymer, wherein the at least one polymer has an average molecular weight between 10.000 and 50.000 Da, between 10.000 and 20.000 Da, between 20.000 and 30.000 Da, between 30.000 and 40.000 Da, between 40.000 and 50.000 Da, including any range or value therebetween. In some embodiments, the composition (e.g. a fiber) comprises a first polymer and at least one polymer select from the second polymer and the third polymer, wherein the at least one polymer has an average molecular weight of at least 5.000 Da, at least 7.000 Da, at least 8.000 Da, at least 9.000 Da, at least 10.000 Da, at least 12.000 Da, at least 15.000 Da, at least 20.000 Da, including any range or value therebetween.

In some embodiments, the third polymer is a branched polymer. In some embodiments, a branched polymer is as described herein above.

In some embodiments, the third polymer has reactivity to the second polymer. In some embodiments, the third polymer comprises a reactive group being capable of covalent bond formation with the second polymer. In some embodiments, the reactive group of the third polymer (e.g. a nucleophilic group) is capable of covalent bond formation with the reactive group of the second polymer (e.g. an electrophile). In some embodiments, the reactive group of the third polymer is capable of covalent bond formation with the tissue-adhesive group of the second polymer.

In some embodiments, the reactive group of the third polymer is selected from the group consisting of: a nucleophilic group (e.g. an amine, a thiol, a phosphine, a hydroxyl), a diene, tetrazine, and azide, or any combination thereof. In some embodiments, the reactive group of the third polymer is a nucleophilic group.

In some embodiments, covalent bond formation is referred to cross-linking.

In some embodiments, cross-linking is inter-crosslinking. As defined herein, the term “inter” refers to the formation of a bond between two reactive groups residing in two polymeric chains, as oppose to the formation of an “intra” bond between two reactive groups residing within the same polymeric chain.

In some embodiments, the second and the third polymer are at least partially crosslinked, so as to form a cross-linked polymer. In some embodiments, the cross-link is formed by reacting the tissue-adhesive group and the reactive group of the third polymer. In some embodiments, cross-link is formed via a “click reaction”, such as azide alkyne cycloaddition, or a reversed order Diels-Alder reaction. In some embodiments, the cross-link is formed by reacting the tissue-adhesive group and the nucleophilic group of the third polymer. In some embodiments, cross-linking is via an amide bond formed by reacting an amino group of the third polymer with NHS of the second polymer. In some embodiments, cross-linking is via a thioester bond, formed by reacting a thiol group of the third polymer with NHS of the second polymer.

In some embodiments, the cross-linked polymer is characterized by a crosslinking degree ranging from 1% to 80%, from 1% to 10%, from 10% to 20%, from 20% to 30%, from 30% to 40%, from 40% to 50%, from 50% to 60%, from 60% to 70%, from 70% to 80%, including any range or value therebetween.

In some embodiments, crosslinking degree of the second and the third polymer is at most 80%, at most 60%, at most 50%, at most 40%, at most 30%, at most 20%, at most 10%, including any range or value therebetween.

In some embodiments, crosslinking is formed in-situ by contacting (i) a composition (e.g. a fiber) comprising a first polymer and one of the second polymer and of the third polymer, with (ii) a composition comprising a complementary polymer (e.g. the second polymer and of the third polymer respectively), as described herein. In some embodiments, the composition (e.g. a fiber) comprising (i) a first polymer and (ii) one of the second polymer and of the third polymer is substantially devoid of crosslinks.

In some embodiments, crosslinking degree of the second polymer with the third polymer is sufficient so as to allow formation of a polymeric fiber, by any one of the fiber manufacturing processes disclosed herein, such as electrospinning. In some embodiments, crosslinking degree of the second polymer with the third polymer is sufficient so as to form a stable composition (e.g. a fiber, a matrix or a layer comprising a plurality of fibers). In some embodiments, crosslinking degree of the second polymer with the third polymer is sufficient so as to form a composition (e.g. a fiber, a matrix or a layer comprising a plurality of fibers) characterized by a sufficient adhesion strength, as described herein.

In some embodiments, at least a portion of the tissue adhesive groups of the second polymer remains unreacted, so as to provide a sufficient amount of binding sites (e.g. covalent bonds) with the biological surface (e.g. a tissue). In some embodiments, at least a portion of tissue adhesive group remains unreacted (e.g. non-crosslinked), so as to establish binding and/or adhesion to a biological surface. In some embodiments, at least a portion of tissue adhesive group remains unreacted, so as to establish sufficient adhesion strength, as described herein. In some embodiments, at least 10, at least 20, at least 30, at least 40, at least 50, at least 60 mol % of the tissue adhesive groups within the composition (e.g. fiber) are unreacted (e.g. intact).

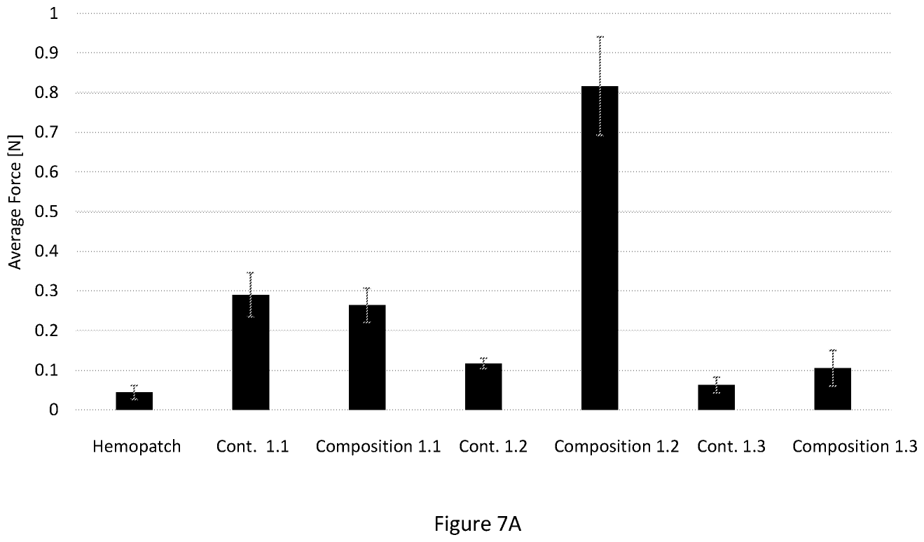

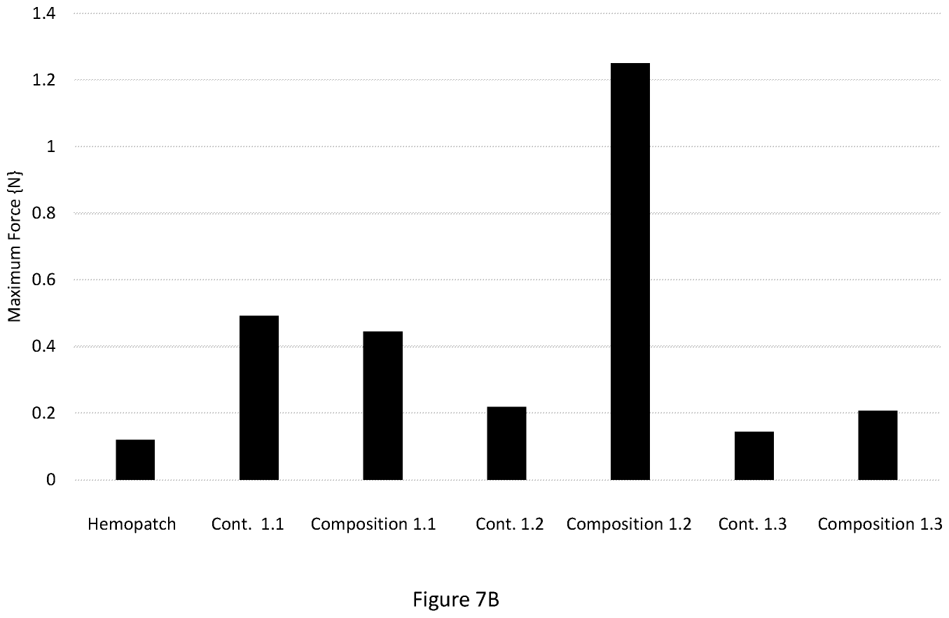

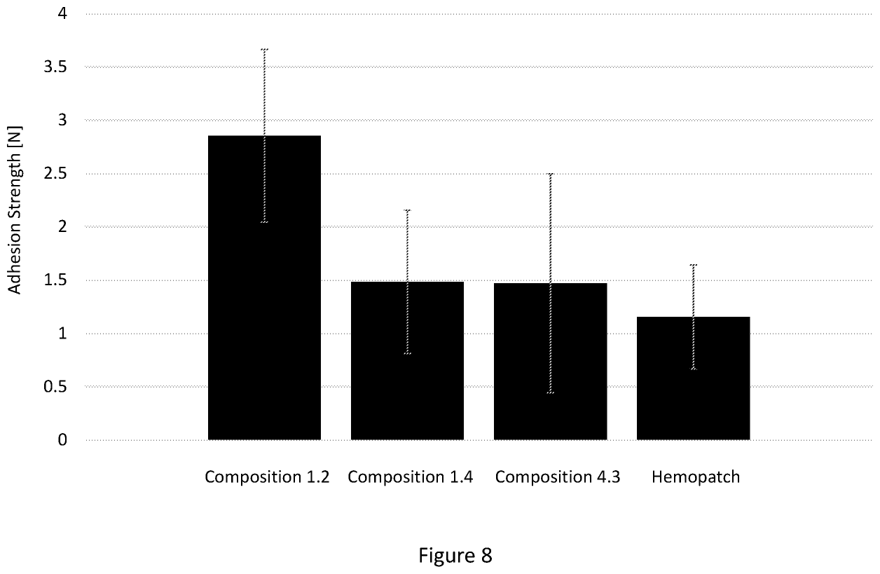

The cross-linked polymer has several advantages over a non-cross-linked polymer. As shown in FIGS. 7 and 8, a polymeric fiber comprising the cross-linked polymer (Compositions 1.2 and 1.4) and (Composition 2, not shown) exhibited the highest adhesion strength, as compared a polymeric fiber comprising a non-crosslinked polymer (Composition 1.1, Control 1.1). Without being limited by any particular mechanism or theory, the enhanced adhesion strength may be related to a mesh-like structure of the cross-linked polymer, and optionally to a beneficial orientation of the tissue-adhesive groups towards tissue contacting outer layer of the polymeric fiber.

In some embodiments, the cross-linked polymer (e.g. partially cross-linked polymer) undergoes an additional crosslinking upon contacting with a biological surface. In some embodiments, the cross-linked polymer further forms a gel upon contacting with a biological surface. In some embodiments, gel formation further attributes to the enhanced adhesion strength of a matrix comprising the cross-linked polymer. In some embodiments, the cross-linked polymer is characterized by a tensile strength and an adhesive strength greater than a tensile strength and an adhesive strength of the non cross-linked polymer.

In some embodiments, the weight per weight (w/w) ratio of the third polymer to the second polymer within the composition ranges from 10:1 to 1:10, from 10:1 to 8:1, from 8:1 to 6:1, from 6:1 to 4:1, from 4:1 to 2:1, from 2:1 to 1:1, from 1:1 to 1:1.5, from 1:1.5 to 1:2, from 1:2 to 1:2.5, from 1:2.5 to 1:3, from 1:3 to 1:5, from 1:1 to 1:5, from 1:1 to 1:4, from 1:1 to 1:3, from 1:1 to 1:2, from 1:5 to 1:7, from 1:7 to 1:10, including any range or value therebetween.

In some embodiments, the weight per weight (w/w) ratio of the second polymer to the third polymer within the composition ranges from 0.8:1 to 1:10, from 0.8:1 to 1:1, from 1:1 to 1:1.5, from 1:1.5 to 1:2, from 1:2 to 1:2.5, from 1:2.5 to 1:3, from 1:3 to 1:5, from 1:1 to 1:5, from 1:1 to 1:4, from 1:1 to 1:3, from 1:1 to 1:2, from 1:5 to 1:7, from 1:7 to 1:10, including any range or value therebetween.

In some embodiments, the w/w ratio of the third polymer to the second polymer within the composition is between 1:1 and 1:2, between 1:1 and 1:1.2, between 1:1.2 and 1:1.5, between 1:1.5 and 1:1.7, between 1:1.7 and 1:2, between 1:2 and 1:3, between 1:3 and 1:5, between 1:5 and 1:10, including any range or value therebetween.

In some embodiments, the composition being characterized by a sufficient adhesive strength and/or sufficient mechanical property comprises a w/w ratio of the third polymer (e.g. PEG-SH and/or PEG-NH₂) to the second polymer (e.g. PEG-NHS) of between 1:1 and 1:2, between 1:1 and 1:1.2, between 1:1.2 and 1:1.5, between 1:1.5 and 1:1.7, between 1:1.7 and 1:2, including any range or value therebetween. In some embodiments, sufficient adhesive strength and/or mechanical property is as described herein.

In some embodiments, the composition being characterized by an adhesive strength of greater than 1.1N, comprises a w/w ratio of the third polymer (e.g. PEG-SH and/or PEG-NH₂) to the second polymer (e.g. PEG-NHS) of between 1:1 and 1:2.

In some embodiments, a molar ratio of the third polymer to the second polymer within the composition ranges from 0.8:1 to 1:10, from 0.8:1 to 1:1, from 1:1 to 1:1.5, from 1:1.5 to 1:2, from 1:2 to 1:2.5, from 1:2.5 to 1:3, from 1:3 to 1:5, from 1:1 to 1:5, from 1:1 to 1:4, from 1:1 to 1:3, from 1:1 to 1:2, from 1:5 to 1:7, from 1:7 to 1:10, including any range or value therebetween. In some embodiments, the molar ratio of the third polymer to the second polymer within the composition is between 1:1 and 1:2. In some embodiments, the composition being characterized by an adhesive strength of greater than 1.1N, comprises a molar ratio of the third polymer (e.g. PEG-SH, and/or PEG-NH₂) to the second polymer (e.g. PEG-NHS) of between 1:1 and 1:2, between 1:2 and 1:3, between 1:3 and 1:5, between 1:5 and 1:10 including any range or value therebetween.

It should be noted, that the molar ratio of the third polymer to the second polymer is maintained so as to assure a molar excess of the tissue-adhesive groups over the reactive groups of the third polymer. In some embodiments, the molar excess is at least 10 mol %, at least 20 mol %, at least 30 mol %, at least 40 mol %, at least 50 mol %, at least 70 mol %, at least 90 mol %, at least 100 mol %, at least 150 mol %, at least 200 mol %, at least 300 mol %, at least 400 mol %, at least 500 mol %, including any range or value therebetween.

Such molar excess is required in order to ensure that at least a part of the tissue-adhesive groups is unreacted, thus allowing to retain the tissue adhesive properties of the second polymer. According to experimental data obtained by the inventors, a composition comprising polymeric fibers composed of PEG-NHS and of PEG-SH at a w/w ratio between PEG-NHS to PEG-SH of 2:1, exhibited preferable adhesion strength and stability compared to polymeric fibers composed of PEG-NHS and of PEG-SH at a w/w ratio between PEG-NHS to PEG-SH of 1:1.

In some embodiments, the w/w ratio of the first polymer to the second polymer within the composition ranges from 1:1 to 20:1, from 1:1 to 3:1, from 3:1 to 5:1, from 5:1 to 8:1, from 8:1 to 10:1, from 10:1 to 15:1, from 15:1 to 20:1, or any range their between.

In some embodiments, a combined w/w content of the second polymer and of the third polymer within the composition is between 20 and 60%, between 20 and 30%, between 30 and 40%, between 40 and 50%, between 30 and 55%, between 50 and 60%, between 50 and 55%, between 55 and 60%, including any range or value their between.

In some embodiments, a combined w/w content of the second polymer (e.g. a polyether-NHS) and of the third polymer (e.g. a polyether-SH, a polyether-NH₂ or both) within the composition is at least 30%, at least 40%, at least 50%, including any range or value therebetween.

In some embodiments, the composition (e.g. in a form of a fiber) comprising the combined w/w content of the second polymer and of the third polymer, as described hereinabove is characterized by an appropriate adhesion strength so as to establish binding and/or adhesion to a biological surface.

In some embodiments, the composition (e.g. the composition of the invention in a form of a fiber) having a combined w/w content of the second polymer and of the third polymer being between 20 and 70%, between 30 and 50%, between 40 and 50% is characterized by a sufficient adhesion strength (e.g. above 1.1N), so as to establish binding and/or adhesion to a biological surface, wherein the adhesion strength is as described herein.

In some embodiments, the composition (e.g. in a form of a fiber) having a combined w/w content of the second polymer and of the third polymer being between 20 and 60%, is characterized by adhesion strength of at least 1N, at least 1.1N, at least 1.2N, at least 1.3N, at least 1.4N, at least 1.5N, at least 1.6N, at least 1.7N, at least 1.8N, at least 1.9N, at least 2N, at least 2.2N, at least 2.4N, at least 2.5N, at least 2.8N, at least 3N, at least 3.2N, at least 4N, including any range or value therebetween, wherein the adhesion strength is as described herein.

In some embodiments, the composition of the invention is characterized by an enhanced mechanical strength (e.g. tensile strength of above 0.3 MPa, above 0.5 MPa, above 0.7 MPa, above 0.9 MPa, above 1 MPa, above 1.5 MPa, above 2 MPa, above 2.5 MPa, above 3 MPa, above 3.5 MPa, above 4 MPa, including any range or value therebetween) compared to a control (e.g. commercially available products), as exemplified herein.

In some embodiments, the composition (e.g. in a form of a fiber) having a combined w/w content of the second polymer and of the third polymer being between 20 and 60%, between 30 and 50%, between 40 and 50% is characterized by an enhanced mechanical strength (e.g. tensile strength of above 0.3 MPa, above 0.5 MPa, above 0.7 MPa, above 0.9 MPa, above 1 MPa, above 1.5 MPa, above 2 MPa, above 2.5 MPa, above 3 MPa, above 3.5 MPa, above 4 MPa, including any range or value therebetween) compared to a control (e.g. commercially available products), as exemplified herein.

In some embodiments, the composition of the invention is a solid. In some embodiments, the composition of the invention (e.g. a solid composition) is substantially devoid of a solvent. In some embodiments, the composition of the invention comprises trace amounts of a residual solvent. In some embodiments, the composition of the invention (e.g. a solid composition) comprises less than 5%, less than 3%, less than 2%, less than 1%, less than 0.5%, less than 0.1%, less than 0.05%, less than 0.01% w/w of an organic solvent. In some embodiments, the composition of the invention is in a form of a fiber. In some embodiments, the composition of the invention is in a form of a matrix comprising a plurality of fibers (as described herein). In some embodiments, the composition of the invention is in a form of a fibrous mat. In some embodiments, the composition of the invention is in a form of a semi-solid or a semi-liquid. In some embodiments, the composition of the invention is in a form of a gel. In some embodiments, the composition of the invention (e.g. a liquid or a semi-liquid) is substantially homogenous. In some embodiments, the composition of the invention is substantially stable, wherein stable is refers to the ability of the composition maintain its structural and/or functional properties (such as a mechanical property, adhesive property, etc.).

It should be understood that the term “semi-liquid” or “semi-solid”, is intended to mean materials which are flowable under pressure and/or shear force. In some embodiments, semi-liquid compositions include creams, ointments, gel-like materials and other similar materials. In some embodiments, the composition is a semi-liquid composition, characterized by a viscosity in a range from 31,000-800,000 cps.

In some embodiments, the composition further comprises a solvent. In some embodiments, a solvent is an organic solvent. In some embodiments, a solvent is an aqueous solvent. In some embodiments, the composition of the invention is a liquid composition. In some embodiments, the composition or the liquid composition comprises a solvent and the fiber, wherein the fiber is as described herein. In some embodiments, the composition comprising a solvent and the fiber is a semi-solid composition, or a semi-liquid composition (e.g. a gel). In some embodiments, a w/w concentration of a solvent within the composition is between 5 and 95%, between 5 and 10%, between 10 and 20%, between 20 and 30%, between 30 and 50%, between 50 and 70%, between 70 and 95%, including any range or value therebetween.

Non-limiting examples of organic solvents include, but are not limited to: alcohols (e.g., methanol, ethanol), hydrocarbons such as alkanes (e.g., hexane), alkenes and alkynes, ethers (e.g. tetrahydrofuran, dioxane), esters, ketones, oils, polar solvents (e.g. dimethylformamide), and non-polar solvents (e.g. chloroform).

In some embodiments, the composition comprises a plurality of solvents.

In some embodiments, the composition comprising the first, and at least one of (i) the second polymer and (ii) the third polymer in form of a blended polymeric fiber (also referred to as: “polymeric fiber”). In some embodiments, the polymeric fiber comprises the first polymer, the second polymer and the third polymer, as described herein.

In some embodiments, at least 20 weight percent (by dry weight) of the polymeric fiber consists of one or more polymers of the invention. In some embodiments, at least 30 weight percent (by dry weight) of the polymeric fiber consists of one or more polymers of the invention. In some embodiments, at least 40 weight percent (by dry weight) of the polymeric fiber consists of one or more polymers of the invention. In some embodiments, at least 50 weight percent (by dry weight) of the polymeric fiber consists of one or more polymers of the invention. In some embodiments, at least 60 weight percent (by dry weight) of the polymeric fiber consists of one or more polymers of the invention. In some embodiments, at least 70 weight percent (by dry weight) of the polymeric fiber consists of one or more polymers of the invention. In some embodiments, at least 80 weight percent (by dry weight) of the polymeric fiber consists of one or more polymers of the invention. In some embodiments, at least 90 weight percent (by dry weight) of the polymeric fiber consists of one or more polymers of the invention.

The term “fiber”, as used herein, describes a class of structural elements, similar to pieces of thread, that are made of continuous filaments and/or discrete elongated pieces. In some embodiments, the first polymer provides or enhances stability of the polymeric fiber. In some embodiments, the polymeric fiber comprising the first polymer, and at least one of (i) the second polymer and (ii) the third polymer has an enhanced stability, compared to a fiber being substantially devoid of the first polymer. In some embodiments, the polymeric fiber is referred to as stable, if the fiber maintains substantially its structure. In some embodiments, maintains substantially is over a time period of at least 1 day (d), at least 10 d, at least 20 d, at least 30 d, at least 50 d, at least 100 d, at least 200 d, at least 300 d, at least 1 year (y), at least 2 y, at least 3 y, including any range or value therebetween. In some embodiments, the fiber is referred to as stable, if it remains structurally intact under physiological conditions (e.g., is not degraded in vivo, and hence is non-biodegradable or non-biocleavable). In some embodiments, the fiber is referred to as stable, if it remains structurally intact under ambient conditions (such as a temperature between 10 and 60° C. and moisture content of between 10 and 99% including any value therebetween).

In some embodiments, the polymeric fiber further comprises an additional biodegradable polymer.

In some embodiments, the polymeric fiber is biodegradable. In some embodiments, the polymeric fiber is characterized by an average fiber diameter of 0.5 to 10 um, from 0.5 to 1.5 um, from 1 to 4 um, from 2 to 4 um, from 4 to 5 um, from 5 to 6 um, from 6 to 7 um, from 7 to 8 um, from 8 to 10 um, or any range therebetween.

In some embodiments, the polymeric fiber is characterized by a melting point of 50 to 150° C., from 50 to 70° C., from 70 to 100° C., from 100 to 120° C., from 120 to 150° C.

As used herein, a melting point or a glass transition temperature is preferably determined according to differential scanning calorimetry, using procedures accepted in the art for such a purpose, using cooling and heating rates of 10° C. per minute. The glass transition typically appears as an intersection between two linear regions in a plot of heat capacity as a function of temperature.

In some embodiments, the polymeric fiber is woven or non-woven. Many suitable techniques will be known to the skilled practitioner for spinning fibers.

In some embodiments, the polymeric fiber is non-woven.

In some embodiments, the polymeric fiber is electrospun.

Without being bound by any particular theory, it is believed that electrospun fibers, and structurally similar fibers, are particularly suitable for forming tissue adhesive layers such as described herein below. In particular, layers of electrospun fibers can be prepared from a wide variety of materials, and allow control over pore size, fiber size, fiber alignment, hydrophobicity, elasticity and mechanical strength.

In some embodiments, the polymeric fiber (e.g. the fiber of the invention) further comprises an additive. In some embodiments, the composition (e.g. the composition of the invention) further comprises an additive. In some embodiments, the composition (e.g. the liquid composition and/or the semi-liquid composition) further comprises an additive. In some embodiments, a w/w concentration of the additive within the composition of the invention is between 5 and 95%, between 5 and 10%, between 10 and 20%, between 20 and 30%, between 30 and 50%, between 50 and 70%, between 70 and 95%, including any range or value therebetween.

Examples of additives include, without limitation, adhesive materials, non-adhesive materials (e.g., materials characterize by particularly low adherence to tissue and/or other substrate), hydrophobic polymer particles, biological and/or bio-active materials, cellular components (e.g., a cell signaling protein, an extracellular matrix protein, a cell adhesion protein, a growth factor, protein A, a protease and a protease substrate), growth factors and therapeutically active agents.

Other additives (e.g., therapeutically active agents) which can be beneficially incorporated into the polymeric fiber and/or into a composition of the invention (e.g. a liquid or a semi-liquid composition) include both natural and/or synthetic polymeric (macro-biomolecules, for example, proteins, enzymes) and non-polymeric (small molecule therapeutics) natural or synthetic agents.

Examples of suitable therapeutically active agents include, without limitation, anti-proliferative agents, cytotoxic factors or cell cycle inhibitors, including CD inhibitors, such as p53, thymidine kinase (“TK”) and other agents useful for interfering with cell proliferation.

Examples of therapeutically active agents that inhibit cell proliferation and/or angiogenesis (antiproliferative drugs) which are particularly useful in drug-eluting systems destined for anticancer treatment, include paclitaxel, sirolimus (rapamycin), farnesylthiosalicylate (FTS, salirasib), fluoro-FTS, everolimus, zotarolimus, daunorubicin, doxorubicin, N-(5,5-diacetoxypentyl)doxorubicin, anthracycline, mitomycin C, mitomycin A, 9-amino camptothecin, aminopertin, antinomycin, N⁸-acetyl spermidine, 1-(2-chloroethyl)-1,2-dimethanesulfonyl hydrazine, bleomycin, tallysomucin, etoposide, camptothecin, irinotecan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine, vinblastine and derivatives thereof.

Additional therapeutically active agents which can be beneficially incorporated into the polymeric fiber and/or into a composition of the invention (e.g. a liquid or a semi-liquid composition) include antibiotic agents. Non-limiting examples of suitable antibiotic agents include gentamicin, ceftazidime, mafenide benzoyl peroxide, octopirox, erythromycin, zinc, silver, tetracyclin, triclosan, azelaic acid and its derivatives, phenoxyethanol and phenoxypropanol, ethyl acetate, clindamycin and meclocycline; sebostats such as flavinoids; alpha and beta hydroxy acids; polydiallyldimethylammonium chloride and bile salts such as scymnol sulfate and its derivatives, deoxycholate and cholate.

Additional therapeutically active agents which can be beneficially incorporated into the polymeric fiber and/or into a composition of the invention (e.g. a liquid or a semi-liquid composition) include analgesic agents, anaesthetic agents, pain-killers, pain-reducers and the like (including NSAIDs, COX-2 inhibitors, K+ channel openers, opiates and morphinomimetics); and hemostatic agents and antihemorrhagic agents.

### Matrix

In some embodiments, provided herein a matrix comprising a tissue adhesive layer. In some embodiments, the tissue adhesive layer comprises a plurality of blended polymeric fibers, wherein the blende polymeric fibers are as described herein above.

In some embodiments, the tissue adhesive layer provides bioadhesive properties to the matrix. In some embodiments, the tissue adhesive layer forms covalent or non-covalent interactions with the tissue, resulting in tissue adherence of the matrix.

In some embodiments, the bioadhesive properties of the tissue adhesive layer are enhanced upon hydration, for example, upon contact with moist tissue.

In some embodiments, tissue adhesive layer facilitates cell attachment and/or proliferation.

As used herein, the term “matrix” refers to one or more layers of polymeric fibers. Matrix may further include any materials incorporated within and/or interposed between the layers. In some embodiments, the terms “matrix” and “tissue adhesive layer” are used herein interchangeably.

In some embodiments, the matrix is a multi-layer matrix, comprising a tissue adhesive layer and an additional layer.

In some embodiments, an additional layer is an elastic layer or a viscoelastic layer. In some embodiments, the additional layer enhances a stability of the tissue-adhesive layer. In some embodiments, stability is as described herein. In some embodiments, the additional layer enhances a mechanical strength of the tissue-adhesive layer. In some embodiments, the additional layer enhances at least one mechanical property of the matrix. In some embodiments, the at least one mechanical property is selected from the group consisting of: Young's modulus, tensile strength, fracture strain, yield point, toughness, work to failure, impact strength, tear strength, flexural modulus, flexural strain and stress at a specific percentage elongation, and abrasion.

In some embodiments, an additional layer is attached to the tissue-adhesive layer, or interposed between two tissue-adhesive layers.

As used herein, the term “elastic layer” refers to a layer of material, wherein the layer exhibits elasticity. Herein, the terms “elasticity” and “elastic” are as described herein above.

As used herein, the term “viscoelastic layer” refers to a layer of material, wherein the layer exhibits viscoelasticity.

An elastic layer according to any one of the embodiments described in this section described in this section may be combined with a viscoelastic polymeric material and/or viscoelastic layer according to any one of the respective embodiments described herein.

As used herein, the term “multi-layer” refers to a presence of at least two distinct layers. The distinct layers may differ, for example, in chemical composition, molecular configuration (e.g., degree and type of crystallinity), physical structure and/or mechanical properties.

As exemplified herein below (Examples section), a matrix, such as described herein, can be formed from biodegradable and biocompatible materials, while exhibiting considerable mechanical strength, high adhesion strength, a high degree of elasticity and flexibility, high porosity (which may support cell growth and tissue adhesion), and a high degree of water-impermeability suitable for creating a tight seal, joining a tissue surface to another tissue, preventing fluid leakage, and preventing bacterial and viral infections.

In some embodiments, the tissue-adhesive layer has a thickness in a range of from 0.5 to 200 μm, from 0.5 to 1 μm, from 1 to 100 μm, from 1 to 5 μm, from 5 to 10 μm, from 10 to 20 μm, from 20 to 30 μm, from 30 to 50 μm, from 50 to 70 μm, from 50 to 100 μm, from 70 to 100 μm, from 100 to 150 μm, from 150 to 200 μm, from 200 to 250 μm, including any range or value therebetween.

In some embodiments, the tissue-adhesive layer is characterized by a tensile strength of at least 0.05 MPa, at least 0.5 MPa, at least 1 MPa, at least 2 MPa at least 3 MPa, at least 4 MPa, at least 5 MPa, at least 7 MPa, at least 8 MPa, at least 10 MPa. In some embodiments, the tissue-adhesive layer is characterized by a tensile strength of between 0.05 and 1 MPa, between 0.5 and 1 MPa, between 1 and 2 MPa, between 2 and 3 MPa, between 3 and 4 MPa, between 4 and 5 MPa, between 5 and 7 MPa, between 7 and 8 MPa, between 8 and 10 MPa, including any range or value therebetween. In some embodiments, the tissue-adhesive layer is characterized by a tensile strength as exemplified herein.

Tensile properties described herein (e.g., tensile strength) are determined in accordance with ASTM international standard D882-12 for testing tensile properties of thin plastic sheeting. Tensile testing characterizes an amount of tensile stress applied to the tested material as a function of tensile strain (increase in length due to tensile stress, as a percentage of the original length) of the material.

The tensile strength is determined as the maximal stress which can be applied to the tested material, such that any further strain is obtained with reduced stress (a phenomenon known as “necking” or is unobtainable because the tensile stress results in rupture (e.g., tearing, cracking) of the material.

In some embodiments, the tissue-adhesive layer is characterized by an adhesion strength ranging from 10 to 400 KPa, from 10 to 50 KPa, from 20 to 50 KPa, from 50 to 80 KPa, from 80 to 100 KPa, from 100 to 200 KPa, from 200 to 300 KPa, from 300 to 400 KPa.

In some embodiments, the tissue-adhesive layer is characterized by an adhesion strength ranging from 0.1 to 2N, from 0.1 to 0.3N, from 0.3 to 0.5N, from 0.5 to 0.7N, from 0.7 to 0.9N, from 0.9 to 1.0N, from 1.0 to 1.2N, from 1.2 to 1.5N, from 1.5 to 2N, including any range or value therebetween. In some embodiments, adhesion strength is referred to an average peel force or to maximum peel force measured by a peel test, wherein the peel test is as described herein.

In some embodiments, the tissue-adhesive layer is characterized by an adhesion strength ranging from 1 to 5N, from 1 to 1.2N, from 1.2 to 1.4N, from 1.4 to 1.6N, from 1.6 to 2N, from 2 to 2.5N, from 2.5 to 3N, from 3 to 3.5N, from 3.5 to 4N, from 4 to 5N, from 5 to 6N, from 6 to 10N, including any range or value therebetween. In some embodiments, the adhesion strength is measured according to the shear test.

In some embodiments, the tissue-adhesive layer is characterized by an adhesion strength of at least 1N, at least 1.1N, at least 1.2N, at least 1.3N, at least 1.4N, at least 1.5N, at least 1.6N, at least 1.7N, at least 1.8N, at least 1.9N, at least 2N, at least 2.2N, at least 2.4N, at least 2.5N, at least 2.8N, at least 3N, at least 3.2N, at least 4N, at least 5N, at least 6N, at least 8N, at least 10N including any range or value therebetween. In some embodiments, adhesion strength is determined by a shear test, as described herein below.

The adhesion strength is determined by two different methods: a peel test and a shear test, as described herein below (Examples section).

In some embodiments, the tissue-adhesive layer is characterized by a water-permeability of less than 1 ml per hour per cm² upon exposure to an aqueous liquid at a pressure of 40 mmHg. In some such embodiments, the water-permeability is less than 0.3 ml per hour per cm². In some embodiments, the water-permeability is less than 0.1 ml per hour per cm². In some embodiments, the water-permeability is less than 0.03 ml per hour per cm². In some embodiments, the water-permeability is less than 0.01 ml per hour per cm².

In some embodiments, the tissue-adhesive layer further comprises an additive (e.g. pharmaceutically active ingredient). In some embodiments, the additive is as described herein above.

In some embodiments, any one of the tissue-adhesive layer and an additional layer is a porous layer. As used herein, the term “porous layer” refers to a layer which comprises voids (e.g., in addition to polymeric material described herein), for example, the space between the polymeric material is not filled in by an additional substance. However, porous layers may optionally comprise an additional substance in the spaces between the polymeric material, provided that at least a portion of the volume of the voids is not filled in by the additional substance.

Many suitable techniques will be known to the skilled practitioner for preparing a polymeric material in porous form, including, without limitation, various techniques for spinning fibers, use of a gas to form a foam, and drying (e.g., lyophilizing) a suspension of polymeric material.

In some embodiments, the porous layer (e.g., the tissue-adhesive layer), is characterized by a porosity of at least 60% (e.g., from 60 to 99%). In some such embodiments, the porous layer is characterized by a porosity of at least 70% (e.g., from 70 to 99%). In some such embodiments, the porous layer is characterized by a porosity of at least 80% (e.g., from 80 to 99%). In some such embodiments, the porous layer is characterized by a porosity of at least 90% (e.g., from 90 to 99%). In some such embodiments, the porous layer is characterized by a porosity of about 90%.

Herein, the term “porosity” refers to a percentage of the volume of a substance (e.g., tissue adhesive layer described herein) which consists of voids.

In some embodiments, the porous layer (e.g., the tissue-adhesive layer) is characterized by a pore size ranging from 0.5 to 100 um, from 0.5 to 2 um, from 2 to 4 um, from 4 to 6 um, from 6 to 7 um, from 7 to 8 um, from 8 to 10 um, from 10 to 15 um, from 15 to 20 um, from 20 to 30 um, from 30 to 40 um, from 40 to 50 um, from 50 to 70 um, from 70 to 100 um.

In some embodiments, the composition (e.g. the matrix) of the invention is characterized by a low swelling ability. In some embodiments, the composition (e.g. the matrix) of the invention is characterized by swelling of about 10%. The inventors tested the swelling ability of the exemplary compositions of the invention. Even after 8 days the swell volumes of the tested compositions were less than 5%, compared to 65% swell volume for Hemopatch™ (according to the published data). In some embodiments, the composition (e.g. the matrix) of the invention is characterized by swell volume of less than 10%, less than 8%, less than 6%, less than 5%, less than 4%, less than 3%, including any range or value therebetween. In some embodiments, the composition (e.g. the matrix) of the invention is characterized by a significant water uptake ability. In some embodiments, the composition (e.g. the matrix) of the invention is characterized by a water uptake between 5 and 10, between 5 and 6, between 6 and 7, between 7 and 8, between 8 and 10 times of the initial sample weight, including any range or value therebetween.

The water uptake of the exemplary compositions (e.g. matrices) of the invention have been compared to the commercially available Hemopatch™. Tested compositions of the invention exhibited the ability to absorb fluids in the range of 5-7 times of their initial weight. Water uptake is an important property for implants, as they are required to absorb the undesired leakage of bodily fluids within an implant site of the subject. In some embodiments, the tested samples being less than 0.35 mm thick, exhibited a water absorption ability that is similar to the thick (2 mm) commercially available Hemopatch™.

### Kit

In another aspect, there is a kit comprising a blended polymeric fiber and a composition; wherein the blended polymeric fiber comprises a first polymer and a second polymer; wherein the composition comprises a third polymer having reactivity to the second polymer; and wherein any one of the second polymer and the third polymer comprises a tissue-adhesive group. In some embodiments, the second polymer and the third polymer of the kit comprises the second branched polymer of the invention or the third polymer of the invention respectively. In some embodiments, the blended polymeric fiber of the kit comprises a first polymer and a second branched polymer of the invention; and the composition of the kit comprises a third polymer of the invention. In some embodiments, the blended polymeric fiber of the kit comprises a first polymer and a third polymer of the invention and the composition of the kit comprises the second branched polymer of the invention.

In some embodiments, the blended polymeric fiber of the kit comprises the second branched polymer of the invention; and the composition of the kit comprises the third polymer of the invention. In some embodiments, the blended polymeric fiber of the kit comprises the third polymer; and the composition of the kit comprises the second branched polymer.

In some embodiments, the composition of the kit further comprises an agent comprising a carrier, an additive, a solvent or any combination thereof, wherein a w/w concentration of the agent within the composition is between 5 and 95%, between 5 and 10%, between 10 and 20%, between 20 and 30%, between 30 and 50%, between 50 and 70%, between 70 and 95% by weight, including any range or value therebetween. In some embodiments, the carrier, the additive, and the solvent are as described herein.

In some embodiments, the composition of the kit is a liquid, wherein the liquid is as described herein. In some embodiments, the composition of the kit is a semi-liquid (e.g. gel), wherein the semi-liquid is as described herein. In some embodiments, the composition of the kit is a solid. In some embodiments, the composition of the kit is substantially homogenous. In some embodiments, the kit of the invention (e.g. the fiber and/or the composition) is substantially stable, wherein stable is as described herein.

In some embodiments, the composition of the kit of the invention comprises an aqueous solution or any other pharmaceutically acceptable solvent. In some embodiments, the solvent is an alcohol (e.g. ethanol) or a mixture of an aqueous solution and the alcohol. In some embodiments, the polymer of the composition of the kit is in a liquid form. In some embodiments, the polymer of the composition of the kit is in a form of a viscous liquid or semi-liquid. In some embodiments, the polymer of the composition of the kit has a viscosity sufficient for application on top of the fiber of the kit. In some embodiments, the polymer of the composition of the kit is spreadable. In some embodiments, the polymer of the composition of the kit is applied by any of spreading, spraying, casting or by any other method well-known in the art. In some embodiments, the composition of the kit is substantially devoid of any solvent and/or carrier. In some embodiments, the composition of the kit consists essentially of the polymer, wherein the polymer is as described herein.

In some embodiments, the first polymer of the kit comprises or is selected from a polyester, a polyanhydride, a polyacetal, a polyorthoester, a polyurethane, a polycarbonate, a polyphosphazene, a polyphosphoester, a polyether, a silicone, a polyamide, a polysulfone, a polyether ether ketone (PEEK), poly(ethylene glycol), polytetrafluoroethylene, polyethylene, a polysaccharide or a combination or a co-polymer thereof. In some embodiments, the first polymer is the first polymer of the invention.

In some embodiments, the second branched polymer, the third polymer, or both comprise or are selected from a polyether, a polyester, a polydioxanone, a polyphosphoester, a polyurethane, and a polyamide or any combination thereof.

In some embodiments, the second polymer of the kit (e.g. the second branched polymer or the third polymer) has an average molecular weight (MW) of at least 10 kDa, at least 20 kDa, at least 15 kDa, at least 30 kDa, at least 40 kDa, including any range or value therebetween. In some embodiments, the blended polymeric fiber comprising the second polymer of the kit having MW of at least 10 kDa is characterized by an enhanced mechanical property, compared to a blended polymeric fiber comprising a second polymer of the kit having a low-MW. In some embodiments, the low-MW is between 1 and 5 kDa, between 1 and 2 kDa, between 2 and 3 kDa, between 3 and 5 kDa, between 5 and 7 kDa, including any range therebetween.

In some embodiments, a w/w content of the second polymer within the blended polymeric fiber of the kit is at least 20%, at least 30%, at least 40%, at least 50%, including any range or value therebetween.

In some embodiments, a w/w content of the first polymer within the blended polymeric fiber of the kit is at least 20%, at least 30%, at least 40%, at least 50%, including nay range or value therebetween.

In some embodiments, a w/w content of the third polymer within the kit is at least 20%, at least 30%, at least 40%, at least 50%, including any range or value therebetween.

In some embodiments, the second polymer of the kit, the third polymer of the kit, or both comprise polyethyleneglycol. In some embodiments, the second polymer of the kit, the third polymer of the kit, or both comprise polyethyleneglycol and wherein the first polymer is selected from the group consisting of: polylactic acid, poly(L-lactic acid), poly(D-lactic acid), polyglycolic acid, poly(L-glycolic acid), poly(D-glycolic acid), nylon and polycaprolactone or any combination or a co-polymer thereof.

In some embodiments, the second polymer of the kit or the third polymer of the kit comprises a nucleophilic group. In some embodiments, the nucleophilic group is as described herein.

In some embodiments, a weight ratio of the first polymer to the second polymer within the kit ranges from 1:1 to 20:1, from 1:1 to 20:1, from 1:1 to 3:1, from 3:1 to 5:1, from 5:1 to 8:1, from 8:1 to 10:1, from 10:1 to 15:1, from 15:1 to 20:1, or any range their between.

In some embodiments, a molar ratio of the second polymer to the third polymer within the kit is from 1:0.8 to 1:20, from 1:0.8 to 1:1, from 0.8:1 to 1:1, from 1:1 to 1:1.5, from 1:1.5 to 1:2, from 1:2 to 1:2.5, from 1:2.5 to 1:3, from 1:3 to 1:5, from 1:1 to 1:5, from 1:1 to 1:4, from 1:1 to 1:3, from 1:1 to 1:2, from 1:5 to 1:7, from 1:7 to 1:10, from 1:10 to 1:15, from 1:15 to 1:20, including any range or value therebetween, wherein the second branched polymer comprises the nucleophilic group, and the third polymer comprises the tissue-adhesive group.

In some embodiments, a weight ratio of the third polymer to the second polymer within the kit ranges from 1:1 to 1:10, from 1:1 to 1:1.5, from 1:1.5 to 1:2, from 1:2 to 1:2.5, from 1:2.5 to 1:3, from 1:3 to 1:5, from 1:1 to 1:5, from 1:1 to 1:4, from 1:1 to 1:3, from 1:1 to 1:2, from 1:5 to 1:7, from 1:7 to 1:10, including any range or value therebetween. In some embodiments, a weight ratio of the third polymer (e.g. a tissue-adhesive group bearing polymer, such as polyether-NHS) to the second polymer (e.g. nucleophilic group bearing polymer, such as aminated polyether, or thiolated polyether) within the kit is from 1:1 to 1:1.5, from 1:1.5 to 1:2, from 1:2 to 1:2.5, from 1:2.5 to 1:3, including any range or value therebetween.

In some embodiments, the molar ratio of the second polymer (e.g. aminated polyether, or thiolated polyether) to the third polymer (e.g. a polyether-NHS) within the kit is between 1:1 and 1:20, between 1:1 to 1:2, between 1:5 to 1:7, between 1:7 to 1:10, between 1:10 to 1:15, between 1:15 to 1:20, including any range or value therebetween.

In some embodiments, the molar ratio of the third polymer (e.g. aminated polyether, or thiolated polyether) to the second polymer (e.g. a polyether-NHS) within the kit is between 1:1 and 1:20, between 1:1 to 1:2, between 1:5 to 1:7, between 1:7 to 1:10, between 1:10 to 1:15, between 1:15 to 1:20, including any range or value therebetween.

In some embodiments, a polymer comprising the tissue adhesive group is present within the kit of the invention and/or within the composition of the invention in a molar excess relative to a polymer comprising the reactive group (e.g. a nucleophile).

In some embodiments, the blended polymeric fiber in contact with the additional component results in a tissue-adhesive layer. In some embodiments, the tissue-adhesive layer is as described hereinabove. In some embodiments, the tissue-adhesive layer comprises the second branched polymer at least partially crosslinked with the third polymer. In some embodiments, the tissue-adhesive layer comprises the blended polymeric fiber at least partially crosslinked with the third polymer. In some embodiments, crosslinked is as described herein. In some embodiments, crosslinked is by reacting the tissue-adhesive group and the reactive group (e.g. nucleophilic group). In some embodiments, the kit is for utilizing an aminated branched polymer for forming the tissue-adhesive layer (e.g. crosslinked tissue-adhesive matrix), wherein the tissue-adhesive layer is as described herein. In some embodiments, the aminated branched polymer comprises aminated polyether, such as PEG-NH2.

In some embodiments, the third polymer of the composition of the kit (e.g. the second branched polymer or the third polymer of the invention) has MW of less than 10 kDa, of less than 8 kDa, of less than 7 kDa, of less than 6 kDa, of less than 5 kDa, of less than 3 kDa, of less than 2 kDa. In some embodiments, the composition of the kit comprising the third polymer (e.g. the second branched polymer or the third polymer of the invention) having MW of less than 10 kDa, of less than 8 kDa, of less than 7 kDa, of less than 6 kDa, of less than 5 kDa, of less than 3 kDa, of less than 2 kDa, results in a tissue-adhesive layer characterized by an enhanced adhesive strength, compared to a composition comprising a third polymer having MW greater than 10 kDa.

In some embodiments, the tissue-adhesive layer being characterized by an adhesive strength of greater than 1.1N, is formed by contacting the composition of the kit with of the blended polymeric fiber of the kit, wherein a molar ratio of the second polymer (e.g. PEG-SH, and/or PEG-NH₂) to the third polymer (e.g. PEG-NHS) of the kit is between 1:1 and 1:20, between 1:1 to 1:2, between 1:5 to 1:7, between 1:7 to 1:10, between 1:10 to 1:15, between 1:15 to 1:20, including any range or value therebetween.

In some embodiments, a w/w ratio of the second polymer (e.g. PEG-SH, and/or PEG-NH₂) to third polymer (e.g. PEG-NHS) of the kit is between 10:1 and 1:1, between 10:1 to 8:1, between 8:1 to 6:1, between 6:1 to 4:1, between 4:1 to 3:1, between 3:1 to 2:1, between 2:1 to 1:1, including any range or value therebetween.

In some embodiments, the kit of the invention consists essentially of the first polymer, the second branched polymer, and the third polymer. In some embodiments, (i) the first polymer, and (ii) any of the second branched polymer and the third polymer compose at least 80%, at least 85%, at least 90%, at least 95%, at least 97%, at least 98%, at least 99%, at least 99.5%, at least 99.9%, by weight of the blended polymeric fiber of the kit. In some embodiments, the first polymer, the second branched polymer and the third polymer compose at least 80%, at least 85%, at least 90%, at least 95%, at least 97%, at least 98%, at least 99%, at least 99.5%, at least 99.9%, by weight of the polymeric content of the kit and/or of the composition of the invention.

### Use

In some embodiments, the matrix is a medical device. In some embodiments, the medical device is an implantable medical device.

In some embodiments, the medical device is for use in the field of general surgery, neurology, ear-nose and throat, urology, gynecology/obstetrics, thoracic, dental/maxillofacial, gastroenterology, plastic surgery, ophthalmology, cardiovascular and/or orthopedic medicine.

In some embodiments, the matrix as described herein above, is for adhering or sealing of at least one biological surface. In some embodiments, the matrix as described herein above, is for promoting or increasing of bioadhesion or sealing of a biological surface. In some embodiments, the matrix as described herein above, is for promoting or increasing of blood coagulation in a subject in need thereof.

In some embodiments, the matrix is for use in repairing and/or substituting a biological surface.

As used herein, the term “biological surface” refer to any surface comprising cells and/or biological molecules (e.g. proteins, polysaccharides, lipids, nucleic acids). Non-limiting examples of “biological surface” include, but are not limited to: tissue surface, synthetic graft surface, and organ surface.

In some embodiments, a biological surface to be repaired and/or substituted is a soft tissue. In some embodiments, a biological surface to be repaired and/or substituted is a connective tissue. In some embodiments, a biological surface to be repaired and/or substituted is a membrane (e.g., following traumatic injury, hernia and/or surgical incision of the membrane). In some embodiments, the membrane to be repaired and/or substituted is dura mater (e.g., following traumatic injury and/or surgical incision of the dura mater).

In some embodiments, the matrix is for use in bond formation to a biological surface, wherein the biological surface is selected from the group consisting of: a tissue surface, a synthetic graft surface, and an organ surface in a subject in need thereof. In some embodiments, the matrix is for use in joining a tissue surface to another tissue, or for sealing a tissue surface in a subject in need thereof. In some embodiments, the matrix is for use in promoting/enhancing wound healing, in a subject in need thereof. In some embodiments, the matrix is for use in wound closure in a subject in need thereof. In some embodiments, the matrix is for use in sealing of joined tubular structures such as blood vessels in a subject in need thereof. In some embodiments, the matrix is for use in sealing air leaks in the lung in a subject in need thereof.

The matrix, according to the present invention is suitable for application to both internal and external surfaces of the body, i.e. they may be applied topically to the exterior of the body (e.g. to the skin) or to internal surfaces such as surfaces of internal organs exposed during surgical procedures, including conventional and minimally invasive surgery. In some embodiments, the matrix is suitable for sustaining internal surgical incision closure. In some embodiments, the matrix is suitable for surgical applications in the following areas: thoracic and cardiovascular, general surgery, urology, neurosurgery. In some embodiments, the matrix is suitable for preventing or limiting intra and post-surgical bleeding and leakage of bodily fluids, e.g. after hepatobiliary and pancreatic surgery. In some embodiments, the matrix can be applied in the site that requires tissue repair, tissue sealing or other treatments. Furthermore, the materials described in this invention may be also used as coatings, i.e. materials capable of adhering to a surface while forming a layer on it.

In some embodiments, the composition or kit of the present invention can be used for local delivery of drugs or other therapeutic materials into tissues.

It should be noted that the term “adhesive” is used herein to describe materials capable of adhering to surfaces. The term “sealant” is defined as materials capable of adhering to a surface for preventing fluid (such as blood or any other biological fluid) leaks from the surface, in particular from internal tissues or organs, as well as from synthetic grafts and or implants. A sealant is also referred to materials, capable of self-adhering.

Other non-limiting examples of treatments for which the matrix may be used include, without limitation: dural repair, hernia repair, supporting another medical implant (such as in breast reconstruction surgery), sealing an anastomosis, inhibition of post-surgical adhesions between tissues and promotion of hemostasis (e.g., wherein the matrix is coated with thrombin and/or fibrinogen and/or fibrin or a the carrier polymer is composed of material that mechanically promote hemostasis); as well as administration of a therapeutically effective agent (e.g., by incorporating the therapeutically effective agent in and/or on the core matrix, according to any of the embodiments described herein relating to inclusion of an additional ingredient).

In another embodiment, the present invention provides a method for preventing, inhibiting or reducing fibrosis, scarring and/or adhesion in a target site, the method comprising the step of: (a) providing the composition of the invention, (b) applying the composition to the target site, thereby forming, in situ, an adhesion barrier adherent to the target site, thereby preventing, inhibiting or reducing fibrosis, scarring and/or adhesion of traumatized tissues. In some embodiments, the step (b) initiates crosslinking (e.g. between the second branched polymer and the third polymer of the invention), so as to form an adhesive barrier or the matrix of the invention.

In another embodiment, the present invention provides a method for preventing, inhibiting or reducing fibrosis, scarring and/or adhesion in a target site, the method comprising the step of: (a) providing the blended polymeric fiber of the kit, (b) applying the blended polymeric fiber to the target site, and (c) applying the composition of the kit on top of the blended polymeric fiber, thereby forming, in situ, an adhesion barrier adherent to the target site, thereby preventing, inhibiting or reducing fibrosis, scarring and/or adhesion of traumatized tissues.

In another embodiment, the method of the invention further comprising the step of mixing the blended polymeric fiber and the composition of the kit, so as to form a composite prior to applying the composite to the traumatized tissue. According to some embodiments, the mixing step initiates crosslinking (e.g. between the second branched polymer and the third polymer), so as to form an adhesive barrier or the matrix of the invention.

In some embodiments, the target site is a surgical site. In some embodiments, the target site is a post-operative surgical site. In some embodiments, the target site is a biological surface. In some embodiments, the fibrosis, scarring and/or adhesion results from a surgical procedure. In some embodiments, the fibrosis, scarring and/or adhesion results from blunt trauma or a fracture.

Adhesions are known in the art as abnormal, fibrous bands of scar tissue that can form inside the body as a result of the healing process that often follows open or minimally invasive surgical procedure including abdominal, gynecologic, cardiothoracic, spinal, plastic, vascular, ENT, ophthalmologic, urologic, neuro, or orthopedic surgery. Adhesions are typically connective tissue structures that form between adjacent injured areas within the body. Briefly, localized areas of injury trigger a healing response that culminates in healing and scar tissue formation. If scarring results in the formation of fibrous tissue bands or adherence of adjacent anatomical structures (that should normally be separate), adhesion formation is said to have occurred.

Post-surgical adhesions are a consequence resulting when injured or traumatized tissue surfaces, following incision, cauterization, suturing or other mechanical means of trauma, fuse together to form scar tissue. Adhesions can also occur in areas that have undergone blunt trauma or in tissue surrounding fractures. The mechanism of adhesion formation at a traumatized area is based on secretion of a tissue exudate, which in turn induces fibroblast proliferation and consequent formation of collagenous adhesions. These adhesions scar-up the tissue and lead to dysfunctional soft tissues.

Adhesion formation may occur following any surgery or trauma, and is a source of considerable morbidity. For example, postoperative intra-abdominal and pelvic adhesions are a leading cause of infertility, chronic pelvic pain, and intestinal obstruction. Adhesions forming in the tissue may also irritate surrounding nerves and disrupt nerve transmissions, resulting in a significant reduction of sensory or motor function.

In some embodiments, reducing adhesion includes a decrease in adhesion formation and does not require complete alleviation of adhesion signs or symptoms, and does not require a cure. In various embodiments, reducing adhesion formation includes even a marginal decrease in adhesion formation by for example at least 5%, 10%, 15%, 20%, 25%, 30%, 35%, 40%, 45% or higher decreases in adhesion formation or compared to control.

“Reducing adhesions” refers to administering the first and second compositions disclosed herein so as to cause a reduction in the number of adhesions, extent of adhesions (e.g., area), and/or severity of adhesions (e.g., thickness or resistance to mechanical or chemical disruption) relative to the number, extent, and/or severity of adhesions that would occur without such administration. In various embodiments, reducing adhesions may be part of a protocol and also include performing a procedure (e.g., subsequent surgery to reduce adhesions). The compositions or procedure may inhibit formation, or growth of adhesions following an adhesion promoting stimulus, may inhibit progression of adhesions, and/or may inhibit recurrence of adhesions following their spontaneous regression or following mechanical or chemical disruption.

As used herein the term “reducing” in any grammatical form thereof comprises at least 5%, 10%, 15%, 20%, 25%, 30%, 35%, 40%, 45%, 50%, 60%, 70%, 80%, 100%, 200%, 500%, 1000% or higher reduction of one or more value or parameter, including nay range therebetween.

As used herein the term “enhancing” or the term “increasing” in any grammatical form thereof comprises at least 5%, 10%, 15%, 20%, 25%, 30%, 35%, 40%, 45%, 50%, 60%, 70%, 80%, 100%, 200%, 500%, 1000% or higher enhancement of one or more value or parameter, including nay range therebetween.

“Preventing adhesions” refers to administering the first and second compositions prior to formation of adhesions in order to reduce the likelihood that adhesions will form in response to a particular insult, stimulus, or condition. In various embodiments, preventing adhesions may be part of a protocol and also include performing a procedure (e.g., surgery to reduce adhesions). It will be appreciated that “preventing adhesions” does not require that the likelihood of adhesion formation is reduced to zero. Instead, “preventing adhesions” refers to a clinically significant reduction in the likelihood of adhesion formation following a particular insult or stimulus, e.g., a clinically significant reduction in the incidence or number of adhesions in response to a particular adhesion promoting insult, condition, or stimulus.

In various embodiments, the adhesion barrier can act as an adhesion barrier that can be administered or applied to the target tissue site before, during or after the surgery to reduce, prevent or inhibit adhesions. In some embodiments, the adhesion barrier creates a barrier that separates opposing tissue surfaces or tissue-organ surfaces while injured or traumatized tissues heal. In growth of scar tissue and the formation or reformation of adhesions immediately adjacent to the adhesion barrier is thus prevented.

In another embodiment, the target site is a site of tissue injury including, but not limited to, sites of incision, drying, suturing, excision, abrasion, contusion, laceration, anastomosis, manipulation, prosthetic surgery, curettage, orthopedic surgery, neurosurgery, cardiovascular surgery, and plastic or reconstructive surgery. Target sites are also here understood to include neighboring undamaged tissue. In another embodiment, the target site is an area that has been exposed to blunt trauma or the soft tissue surrounding a fracture.

In some embodiments, the invention has application in various surgical procedures. In another embodiment, the surgical procedure is a gynecological surgical procedure (myomectomy via laparotomy or laparoscopy). According to non-limiting embodiments, during removal of a fibroid, an incision is made in the uterus, and a barrier can be formed in between the uterus and the surrounding tissues to prevent adhesion.

In another embodiment, the surgical procedure is abdominal surgery. According to non-limiting embodiments, an adhesion barrier can be used to prevent peritoneal adhesions and therefore prevent intestinal obstruction.

In another embodiment, the surgical procedure is cardiac surgery. According to non-limiting embodiments, a barrier can be used to prevent post-operative adhesion after cardiac procedures.

In another embodiment, the surgical procedure is craniofacial surgery. According to non-limiting embodiments, a barrier can protect the exposed cortex during craniotomy to prevent the skull and the cortex from adhering.

In another embodiment, the surgical procedure is musculoskeletal surgery. According to non-limiting embodiments, a barrier can prevent adherence of a tendon and the surrounding tissues.

In some embodiments, the adhesion barrier is biocompatible. i.e., does not cause substantial tissue irritation or necrosis at the target tissue site.

In some embodiments, the medical device is configured for eluting a therapeutically active agent, e.g., an agent included as an additional ingredient according to any of the respective embodiments described herein. In some such embodiments, the medical device is a stent. Optionally, the composition-of-matter forms at least a portion of a flexible sleeve of the stent.

The therapeutically active agent may optionally be incorporated within the matrix and/or on a surface of the matrix. Optionally, the therapeutically active agent is incorporated within a drug-eluting layer within the matrix and/or on a surface of the matrix. Such a drug eluting layer may be formed of any suitable substance known in the art of drug-eluting layers.

Herein, the phrase “repairing and/or substituting a biological tissue” refers to repair of tissue which is physically damaged in any manner, and encompasses supporting and/or holding damaged tissue together in vivo or ex vivo, as well as filling gaps formed by an absence of tissue (substituting tissue). The damaged tissue may be damaged, for example, by detachment (e.g., tearing, cutting), compressive stress, tensile stress, shear stress, cellular dysfunction and/or cell death.

In some embodiments, there is provided a method of repairing and/or substituting a biological tissue in a subject in need thereof, the method comprising contacting the biological tissue with the matrix (e.g., medical device) described herein above. In some embodiments, the subject is an animal subject. In some embodiments, the subject is a human subject. In some embodiments, the subject is afflicted with a trauma and/or an injury. In some embodiments, the subject undergoes a surgery. In some embodiments, the subject is afflicted with bleeding. In some embodiments, the subject is afflicted with loss of a biological fluid from one or more organs.

In some embodiments, the method comprises affixing at least a portion of the matrix in/on a biological tissue. In some embodiments, the affixing is performed by curing. In some embodiments, the curing is performed via covalent bond formation with the tissue-adhesive layer, as described herein above.

In some embodiments, the method is for adhering or sealing of at least one biological surface.

### Manufacturing Process

In some embodiments, there is provided a process for manufacturing a polymeric fiber, according to any of the respective embodiments described herein. In some embodiments, the process comprises: (i) mixing a solvent with a first polymer, and with at least one of a branched second polymer and a third polymer, thereby obtaining a solution; and (ii) providing the solution into an electrospinning apparatus. In some embodiments, the polymeric fiber is as described hereinabove (e.g. for a composition and/or for a kit of the invention). In some embodiments, the method for manufacturing the polymeric fiber of the composition of the invention comprises (i) mixing a solvent with a first polymer, with a second branched polymer and with a third polymer, thereby obtaining a solution; and (ii) providing the solution into an electrospinning apparatus. In some embodiments, the first polymer, the second branched polymer and the third polymer are as described hereinabove.

In some embodiments, the method for manufacturing the polymeric fiber of the kit of the invention comprises (i) mixing a solvent with a first polymer, and with one of a second polymer and a third polymer, thereby obtaining a solution; and (ii) providing the solution into an electrospinning apparatus, wherein the first polymer, the second branched polymer and the third polymer are as described hereinabove.

In some embodiments, the method for manufacturing the composition of the kit of the invention, comprises providing the second branched polymer or the third polymer and mixing the second branched polymer or the third polymer with a solvent, thereby obtaining the composition of the kit.

In some embodiments, the process further comprises drying of the polymeric fiber. In some embodiments, drying is performed at 10 to 90° C.

In some embodiments, drying comprises vacuum drying. In some embodiments, drying is performed by convection drying, such as by applying a hot gas stream to a fiber surface. In some embodiments, drying is performed by cold drying, such as by applying a de-humidified gas stream to the surface. In some embodiments, drying is performed by infrared (IR) drying. In some embodiments, drying is performed by microwave drying. Generally, the drying method and exact drying conditions selected will depend upon, among other things, chemical and physical properties of the polymer fiber.

In some embodiments, the process is for manufacturing a layer of polymeric fibers (e.g. a tissue-adhesive layer, according to any of the respective embodiments described herein).

Any of the fibers described herein may optionally be produced by any suitable technique for preparing fibers (including macro-sized fibers, micro-sized fibers and nano-sized fibers), such as conventional fiber-spinning techniques. Such techniques include, for example, solution spinning, electrospinning, wet spinning, dry spinning, melt spinning and gel spinning. Each spinning method imparts specific physical dimensions and mechanical properties of the resulting fibers and can be tuned to give the desired characteristics according to the required application of the fibers and layer of fibers described herein.

Briefly, a fiber spinning technique optionally involves the use of spinnerets. These are similar, in principle, to a bathroom shower head, and may have from one to several hundred small holes. As the filaments, or crude fibers, emerge from the holes in the spinneret, the dissolved or liquefied polymer is converted first to a rubbery state and then solidified. This process of extrusion and solidification of “endless” crude fibers is called spinning, not to be confused with the textile operation of the same name, where short pieces of staple fiber are twisted into yarn.

Wet spinning is used for fiber-forming substances that have been dissolved in a solvent. The spinnerets are submerged in a chemical bath and as the filaments emerge they precipitate from solution and solidify. Because the solution is extruded directly into the precipitating liquid, this process for making fibers is called wet spinning. Fibers such as, for example, acrylic, rayon, aramid, modacrylic and spandex can be produced by this process.

Dry spinning is also used for fiber-forming substances in solution, however, instead of precipitating the polymer by dilution or chemical reaction, solidification is achieved by evaporating the solvent in a stream of air or inert gas. The filaments do not come in contact with a precipitating liquid, eliminating the need for drying and easing solvent recovery. This process may be used for the production of, for example, acetate, triacetate, acrylic, modacrylic, PBI, spandex and vinyon.

In melt spinning, the fiber-forming substance is melted for extrusion through the spinneret and then the crude fibers directly solidified by cooling. Melt spun crude fibers can be extruded from the spinneret in different cross-sectional shapes (round, trilobal, pentagonal, octagonal and others). Nylon (polyamide), olefin, polyester, saran and sulfar, for example, are produced in this manner. Non-polymeric fibers can also be produced by melt-spinning.

Gel spinning is a special process used to obtain high strength or other special fiber properties. The polymer is not in a true liquid state during extrusion. Not completely separated, as they would be in a true solution, the polymer chains are bound together at various points in liquid crystal form. This produces strong inter-chain forces in the resulting filaments that can significantly increase the tensile strength of the fibers. In addition, the liquid crystals are aligned along the fiber axis by the shear forces during extrusion. The filaments emerge with an unusually high degree of orientation relative to each other which increases their strength. The process can also be described as dry-wet spinning, since the filaments first pass through air and then are cooled further in a liquid bath. Some high-strength polyethylene and aramid fibers, for example, are produced by gel spinning.

Alternatively, the fibers can be of natural or synthetic origins, and can be provided ready for use without further manipulation or preparation procedures or upon surface treatment thereof.

In some embodiments, the fibers are formed of electrospun polymeric material.

As used herein, the terms “electrospin”, “electrospinning”, “electrospun” and the like refer to a technology which produces fibers (e.g., nanofibers) from a polymer solution. During this process, one or more polymers of the polymeric material as described herein are liquefied (i.e., melted or dissolved) and placed in a dispenser. An electrostatic field is employed to generate a positively charged jet from the dispenser to the collector. Thus, a dispenser (e.g., a syringe with metallic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Reverse polarity for establishing motions of a negatively charged jet from the dispenser to the collector is also contemplated. At the critical voltage, the charge repulsion begins to overcome the surface tension of the liquid drop. The charged jets depart from the dispenser and travel within the electrostatic field towards the collector. Moving with high velocity in the inter-electrode space, the jet stretches and the solvent therein evaporates, thus forming fibers which are collected on the collector, e.g., in a form of a layer of fibers.

Several parameters may affect the diameter of the fiber, these include, the size of the dispensing hole of the dispenser, the dispensing rate, the strength of the electrostatic field, the distance between the dispenser and/or the concentration of the polymeric material used for fabricating the electrospun fiber.

The dispenser can be, for example, a syringe with a metal needle or a bath provided with one or more capillary apertures from which the liquefied polymeric material as described herein can be extruded, e.g., under the action of hydrostatic pressure, mechanical pressure, air pressure and high voltage.

In some embodiments, the collector is a rotating collector which serves for collecting the electrospun fibers thereupon. Employing a rotating collector can result in a layer of electrospun fibers with a continuous gradient of porosity. Such a porosity gradient can be achieved by continuous variation in the velocity of the collector or by a longitudinal motion of the dispenser, these result in a substantial variation in the density and/or spatial distribution of the fibers on the collector and thus, result in a porosity gradient along the radial direction or along the longitudinal direction of the collector, respectively. Typically, but not obligatorily, the rotating collector has a cylindrical shape (e.g., a drum); however, it will be appreciated that the rotating collector can be also of a planar geometry.

In some embodiments, the collector is a flat ground collector which serves for collecting the electrospun scaffold thereupon. Employing a flat ground collector enables collection of random nanofibers. It will be appreciated that the flat ground collector is typically a horizontal collector or a vertical collector.

In some embodiments, any layer of polymeric fibers (including tissue-adhesive layer, according to any of the respective embodiments described herein) is optionally prepared by continuous electrospinning.

In some embodiments, there is provided a process of preparing a matrix according to any of the respective embodiments described herein. In some embodiments, the process comprises the steps of manufacturing a layer of polymeric fibers (according to any of the respective embodiments described herein) and optionally an additional layer by continuous electrospinning, thereby forming the matrix.

In some embodiments, there is provided a process of preparing a multi-layer matrix according to any of the respective embodiments described herein. In some embodiments, the process comprises providing a first layer of polymeric fibers (e.g. a tissue-adhesive layer), placing an additional layer parallel to the first layer, and pressing the first layer and the additional layer together, thereby forming the multi-layer matrix.

In some embodiments, pressing the first layer and the additional layer together comprises applying a pressure of at least 1 gram/cm2. In some embodiments, the pressure is at least 2 gram/cm2. In some embodiments, the pressure is at least 4 gram/cm2. In some embodiments, the pressure is at least 8 gram/cm2.

In some embodiments, the process further comprises heating any one of the layers prior to, concomitantly with, and/or subsequently to pressing the layers. In some embodiments, the heating is to a temperature which is above a glass transition temperature and/or melting point (optionally a glass transition temperature) of the polymeric fiber forming the layer.

### General:

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict. The words “further” and “optionally” may be used interchangeably.

As used herein, the singular form “a” “an” and “the” include plural references unless the context clearly dictates otherwise. For example, the term “a compound” or “at least one compound” may include a plurality of compounds, including mixtures thereof.

As used herein, the term “substantially” is at least 80%, at least 90%, at least 92%, at least 95%, at least 97%, at least 98%, at least 99% by weight of the composition including any range or value therebetween.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treatment” or “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials

Materials utilized for the preparation of exemplary compositions of the invention are summarized in Table 1A and in Table 1B.

TABLE 1A

| Polymer | Abbreviation |
|---|---|
| 70:30 poly(L-lactic acid)/poly(capro-lactone) | PLCL |
| co-polymer | |
| 25:75 poly(DL-lactic acid)/poly(capro-lactone) | PDLC |
| co-polymer | |
| 4 arm Polyethylene Glycol Amine | 4 ArmPEG-NH2 |
| 4 arm Polyethylene Glycol Thiol | 4 ArmPEG-SH |
| 4 arm Polyethylene Glycol Succininmidyl Glutarate | 4 ArmPEG-NHS |
| 8 arm Polyethylene Glycol Succininmidyl Glutarate | 8 ArmPEG-NHS |
| 4 arm Polythylene Glycol Isocyanate | 4ArmPEG- |
| | Isocyanate |
| Methoxy Polyethylene Glycol Succininmidyl | mPEG-NHS |
| Methoxy Polyethylene Glycol Amine | mPEG-NH2 |
| Methoxy Polyethylene Glycol Thiol | mPEG-SH |

TABLE 1B

| Solvent | Abbreviation |
|---|---|
| Di-methylformamide | DMF |
| Tetrahydrofuran | THF |
| Dioxane | Dioxane |

### Methods

1. Morphological Characterization

Characterization of the electrospun samples morphology was obtained by analyzing Scanning Electron Microscopy (SEM) images of the electro-spun samples using ImageJ software; samples were sputter-coated with gold. Images were taken of the external surfaces of the samples with environmental scanning electron microscope (SEM) with tungsten filament (Quonta 200, FEI) between 500 and 8000× magnification.

Fiber diameter and Pore size were measured on ×8000 SEM micrographs using an ImageJ linear measurement tool which was calibrated using the scale bar of the SEM image. The sizes of the fibers and the pores were averaged for each sample (10 fiber and pore measurements were analyzed per image).

2. Mechanical Properties

Tensile properties: The Mechanical properties of each electrospun sample is determined by measuring the Tensile strength of each produced sample in accordance with ASTM D882-12: Standard test method for tensile properties of thin plastic sheeting. The test was carried out using LLOYD LS1 uniaxial tensile machine (equipped with a 100N load cell). Samples were cut in a dog bone configuration and thickness was measured at three points along the neck of the dog bone. The test samples were then mounted on the machine clamps. Each sample was stretched until breakage. The maximal tensile strength was determined.

3. Adhesion Strength

The adhesion strength of each produced sample is determined by two different methods: shear test and peel test. A detailed description of the test conditions is provided herein below.

4. Burst Pressure Strength:

The burst resistance of the adhesive compositions of the invention was also evaluated and compared to available tissue adhesive materials such as Hemopatch™. The test was performed based on ASTM F2392. Collagen strips were used as substrate and were prepared as described in the shear test; 3.0 mm diameter hole was created at the center of each collagen section and prototype sample of 15×15 mm section (n=10) were placed on top of the collagen hole and adhered by pressing prototype sample against the collagen for 15 minutes under 160 gr weight. The bonded/attached sections were then mounted and fixed to the burst fixture centering the hole in the middle and were subjected to constant rate of saline flow as described in the ASTM F2392. The burst strength is defined by the average of maximal pressure required to cause leakage of the sample.

5. Swelling and Water Uptake Properties

Dry patches (a rectangular 4×4 cm diameter) were weighted, and then immersed in water at 37° C. for 1 hr until saturation. The samples were taken out from water, their surfaces are slightly dried using a clean dry paper and are reweighted at each time point. Patches dimensions, in both dry and wet conditions and at each time duration, are measured using a caliper.

Water absorption (%)=(Wf−Wi)/Wi×100%; wherein Wi=initial weight, Wf=final weight (after soaking in water).

### Example 1

### Preparation of Exemplary Compositions and Controls

Blended polymeric fibers (exemplary compositions of the invention and controls) were prepared as follows:

The electro-spinning process was carried out at a temperature of 23±5° C. and 35±10% relative humidity, using a syringe pump, a 22-Gauge needle (inner diameter˜0.51 mm), and a high-voltage (30 kV max) DC supply. Solution flow rate was 2.6 ml/hr under a voltage supply of 6±2 kV, and a tip-to-collector distance of 5-8 cm. Patches were collected on a rotating aluminum vertical wheel with a diameter of 51 mm and 45 mm wide, at 310 rpm. Prepared patches were 200±30 μm thick, and were dried from residual solvents in a vacuum at room temperature for 24 hours.

### Preparation of PLCL Fibers Containing Activated PEG Polymers

Control 1.1: Electrospinning of blended PLCL with methoxy-PEG-NHS (Mw=20K) 1:0.333 (w/w) ratio respectively [6.67E-06, 1.65E-05 mol respectively]. Polymers were dissolved in a 25:25:50 (w/w) mixture of DMF:dioxane:THF to form a final concentration of ˜15% (w/w) PLCL solution at RT.

Control 1.2: Electrospinning of blended PLCL with methoxy-PEG-NHS (20K Mw) and methoxy-PEG-thiol (Mw=20K) 1:0.333:0.167 (w/w/w) ratio respectively [6.67E-06, 1.65E-05, 8.35E-06 mol respectively] as described in Control 1.1.

Control 1.3: Electrospinning of blended PLCL with methoxy-PEG-NHS (20K Mw) and methoxy-PEG-NH2 (Mw=2 KDa) 1:0.333:0.167 [6.67E-06, 1.65E-05, 8.35E-06 mol respectively] ratio respectively as described in Control 1.1.

Composition 1.1: Electrospinning of blended PLCL with 4 Arm-PEG-NHS more preferable 8 arm PEG-NHS (Mw=40K) 1:0.33 ratio respectively [6.67E-06, 8.25E-06 mol respectively] as described in Control 1.1.

Composition 1.2: Electrospinning of blended PLCL with 4 Arm-PEG-NHS more preferable 8 arm PEG-NHS (Mw=40K) and 4 Arm PEG-SH (Mw=20K) 1:0.333:0.167 ratio respectively [6.67E-06, 8.25E-06, 8.35E-06 mol respectively]. Solution and final patches were produced as described in Control 1.1.

Composition 1.3: Electrospinning of blended PLCL with 4 Arm-PEG-NHS more preferable 8 arm PEG-NHS (Mw=40K) and mPEG-NH2 (Mw=2 KDa) 1:0.333:0.167 ratio respectively [6.67E-06, 8.25E-06, 8.35E-05 mol respectively]. Solution and final patches were produced as described in Control 1.1.

Composition 1.4: Electrospinning of blended PLCL with 4 Arm-PEG-NH2 more preferable 8 arm PEG-NH2 (Mw=40K) 2:1 ratio respectively. Solution and final patches to be produced as described in Control 1.1. Final polymers concentration is ˜15%. Patches to be supplied with one or multiple syringes or ampules filled with 4ARM-NHS-2 KDa liquid more preferable 8ARM-NHS-2 KDa to be applied on the tissue in situ.

### Preparation of PDLCL Fibers Containing Activated PEG Polymers

Composition 2.1: Electrospinning of blended PDLCL with 4 Arm-PEG-NHS more preferable 8 arm PEG-NHS (Mw=2 KDa) and 4 Arm-PEG-SH more preferable 8 arm PEG-SH (Mw=20 KDa) 2.5:2.5:1 ratio respectively. Solution and final patches to be produced as described in Control 1.1. Final polymers concentration of ˜25%.

### Preparation of PDLC Films Containing Activated PEG Polymers

Composition 3.1: Spreading of blended PDLC with 4 Arm-PEG-NHS more preferable 8 arm PEG-NHS (Mw=10 KDa) 4:1 ratio solution over a PLCL fibers layer. Final polymers concentration˜20%.

This was carried out by spreading a thin film layer of the polymer mixture over the PLCL layer or attached two-layers of PLCL fibers and 150-microns of PDLC, using thin film applicator. It also could be done by spreading the polymer mixture over release paper and attach to a patch of PLCL fibers or by electrospraying of the blended solution onto the fibers. For better covalent binding between the Activated PEG and the patch of PLCL fibers, the external layer of the PLCL fibers may be chemically functionalized. Chemical activation of external surface of the PLCL fibers can be created by treatment with plasma, ozone, γ-ray, electron beam, laser and UV light. Patches to be dried afterwards from residual solvents in a vacuum at RT for at least 12 hours.

Composition 3.2: Spreading of blended PDLC with 4 Arm-PEG-NHS more preferable 8 arm PEG-NHS (Mw=40 KDa) and 4 Arm PEG-SH (Mw=20 KDa) 20:5:1 ratio solution (˜20%) over the patch of the PLCL fibers or a PLCL fibers layer as described above.

Composition 3.3: Spreading of blended PDLC with 4 Arm-PEG-NHS more preferable 8 arm PEG-NHS (Mw=2 KDa) 1:1 ratio solution (˜20%) over the patch of the PLCL fibers or a PLCL fibers layer as described in Composition 3.1.

Composition 3.4: Spreading of blended PDLC with 4 Arm-PEG-NHS more preferable 8 arm PEG-NHS (Mw=2 KDa) and 4 Arm PEG-SH (Mw=20 KDa) 2.5:2.5:1 ratio solution (˜25%) over the patch of the PLCL fibers as described in Composition 3.1.

Composition 3.5: Spreading of blended PDLC with 4 Arm-PEG-NHS more preferable 8 arm PEG-NHS (Mw=2 KDa) and 4 Arm PEG-SH (Mw=20 KDa) 1.5:1.5:1 ratio solution (˜30%) over the patch of the PLCL fibers as described in Composition 3.1.

Preparation of the PLCL fiber layers coated with activated PEG Polymers

Composition 4.1: Distribution of 4 Arm-PEG-NHS (Mw=40 KDa) more preferable 8 Arm-PEG-NHS (Mw=40 KDa) over the surface of the PLCL fiber patch. This could be done by immersion part of PLCL fiber patch in the PEG polymer melt for a designated time and then cooling down or by distributing the PEG mixture onto the surface of the PLCL fiber patch/onto the surface of another PDLC Film spread over the PLCL fiber patch as described in Composition 3.1. The PEG mixture may be fixed on the surface of the patch/the PDLCL Film by melting e.g. by heating the patch at 40° C. for a 1-2 hrs.

Composition 4.2: Homogeneous distribution of 20 mg 4 ARM-PEG-NHS (40 KDa) more preferable 8 ARM-PEG-NHS (Mw=40 KDa) powder over 2.5 cm×4.0 cm surface as described in Composition 4.1.

Composition 4.3: Homogeneous distribution of 30 mg 4 ARM-PEG-NHS (40 KDa) more preferable 8 ARM-PEG-NHS (Mw=40 KDa) and 4 ARM-PEG-SH (Mw=20 KDa) mixture (2:1) over 2.5 cm×4.0 cm surface as described in Composition 4.1.

Composition 4.4: Homogeneous distribution of 20 mg 4 ARM-PEG-isocyanate (Mw=20 KDa) over 2.5 cm×4.0 cm surface as described in Composition 4.1.

Composition 4.5: Homogeneous distribution of 30 mg 4 ARM-PEG-NHS (Mw=40 KDa) more preferable 8 ARM-PEG-NHS (Mw=40 KDa)+4 ARM-PEG-NH2 (Mw=40 KDa) mixture (2:1) over 2.5 cm×4.0 cm surface as described in Composition 4.1.

Composition 4.6: Mixing of 0.2 ml 4 Arm-PEG-NHS (Mw=2 KDa) more preferable 8 Arm-PEG-NHS (Mw=2 KDa) with 0.1 ml 4 Arm-PEG-SH (Mw=2 KDa) more preferable 8 Arm-PEG-SH (Mw=2 KDa) (2:1 ratio) and casting over 4.0 cm×5.0 cm surface of PLCL fiber patch or over the PDLCL Film of a two-layer patch (fibrous PLCL and PDLC Film).

Composition 4.7: Spreading of solely PDLC (30%) solution over yje PLCL fiber patch as described in Composition 3.1. followed by homogeneous distribution of 20 mg 4 Arm-PEG-NHS more preferable 8 arm PEG-NHS (Mw=40 KDa) powder over 2.5 cm×4.0 cm surface.

Composition 4.8: Spreading of solely PDLC 30% solution over the PLCL fiber patch as described in Composition 3.1. followed by homogeneous distribution of 30 mg of 4 Arm-PEG-NHS more preferable 8 arm PEG-NHS (Mw=40K Da) and 4 Arm PEG-SH (Mw=20 KDa) mixture powder (2:1) over 2.5 cm×4.0 cm surface.

Composition 4.9: Spreading of solely PDLC 30% solution over the PLCL fiber patch as described in Composition 3.1. followed by homogenous distribution of 30 mg 4 Arm-PEG-NHS more preferable 8 arm PEG-NHS (Mw=40 KDA) and 4 Arm PEG-NH2 (Mw=40 KDa) mixture powder (2:1) over 2.5 cm×4.0 cm surface.

Composition 4.10: Spreading of solely PDLC 30% solution over the PLCL fiber patch as described in Composition 3.1. followed by homogeneous distribution of 20 mg 4 Arm-PEG-ISOCYANATE (Mw=20 KDa) powder over 2.5 cm×4.0 cm surface.

Additional exemplary fibers or compositions (Table 2) have been prepared and tested, exhibiting properties such as fiber thickness, pore size, tensile strength and adhesion strength (data not shown) similar to the corresponding fibers or compositions listed hereinabove.

TABLE 2

| Assignment | Polymeric content | Ratio |
|---|---|---|
| Composition 1 | PLCL: 4-8 arm PEG-NHS (MW = 20 kDa) | 3:1 |
| Composition 2 | PLCL: 4-8 arm PEG-NHS (MW = 20 kDa): | 3:1:0.5 |
| | 4-8 arm PEG-SH (MW = 20 kDa) | |
| Composition 3 | PLCL: 4-8 arm PEG-NHS (MW = 20 kDa): | 3:1:0.5 |
| | 4-8 arm PEG-NH₂ (MW = 2 kDa) | |
| Control 1 | PLCL: mPEG-NHS (MW = 20 kDa) | 3:1 |
| Control 2 | PLCL: mPEG-NHS (MW = 20 kDa): | 3:1:0.5 |
| | PEG-SH (MW = 20 kDa) | |
| Control 3 | PLCL: mPEG-NHS (MW = 20 kDa): | 3:1:0.5 |
| | PEG-NH₂ (MW = 2 kDa) | |

Compositions described hereinabove comprising multi arm PEG, are referred to exemplary matrices of the electrospun fibers according to some embodiments of the invention. Controls described hereinabove are referred inter alia to matrices of the electrospun fibers, comprising single arm PEG.

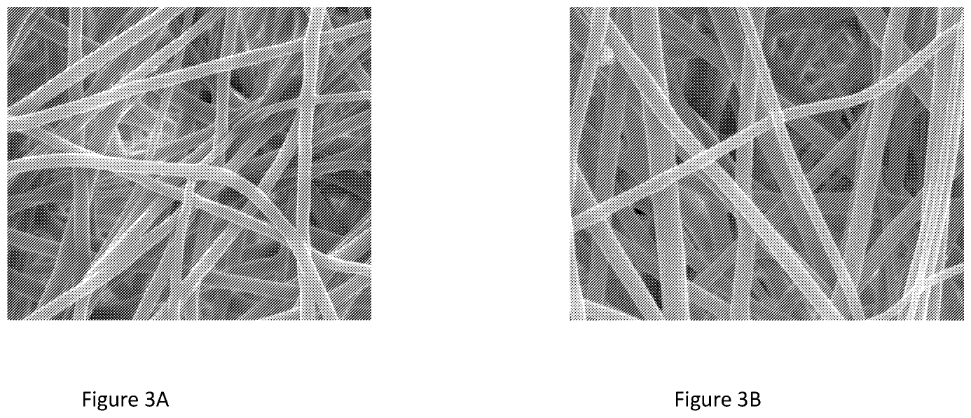

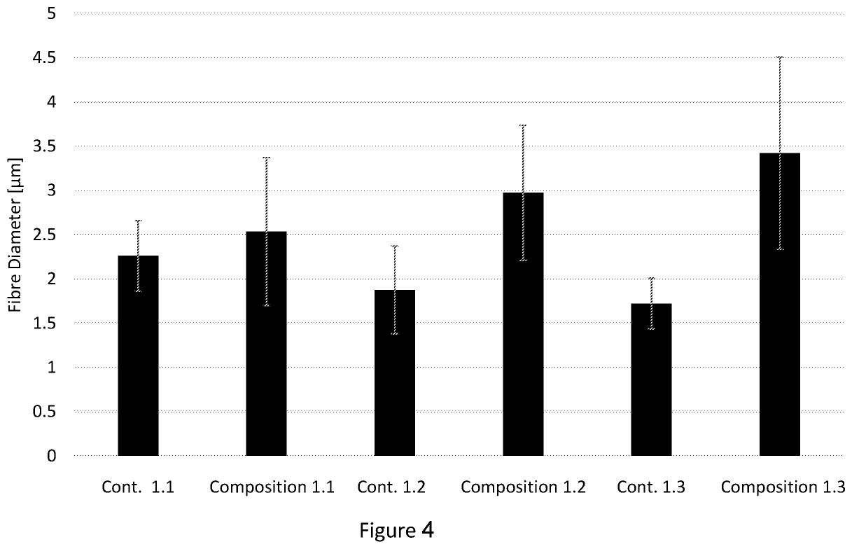

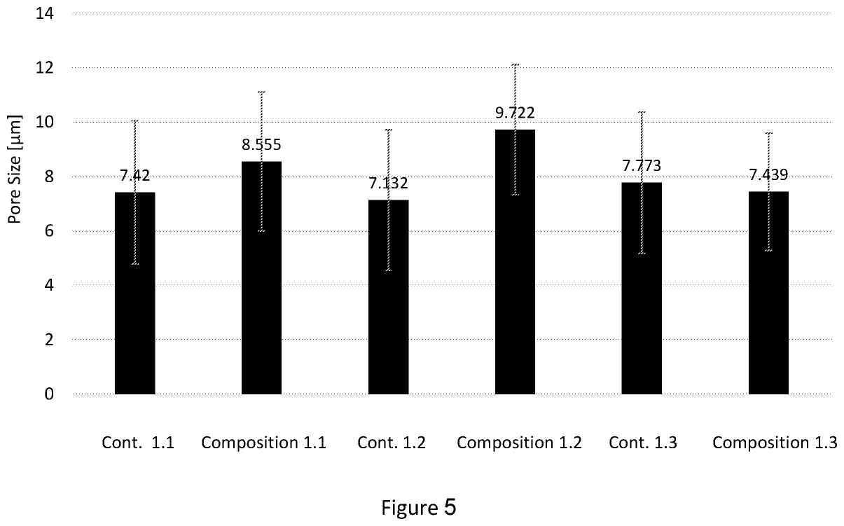

A SEM micrograph, exhibiting a structural image of an exemplary matrix layer of the electrospun fibers of the invention is presented in FIG. 3. SEM images of all samples presented smooth, uniform and bead free fibers with no significant effect of the solution composition of each prototype on fibers morphology. As shown in FIG. 4, electrospun fibers composed of single arm PEG (Controls 1.1-1.3) with the fiber size in a range between 1.08 and 2.7 μm are thinner than electrospun fibers composed of multi arm PEG (Composition 1.1-1.3) with the fiber size in a range between 1.22 and 4.88 μm. On the other hand, the higher fiber diameter of Compositions 1.1-1.3 did not impact the overall pore size of the samples as can be seen in FIG. 5, representing a similar pore size ranging between 7.44 and 9.72 μm for fibers composed of single arm PEG (Controls 1.1-1.3) and for fibers composed of multi arm PEG (Compositions 1.1-1.3).

### Example 2

### Peel Test Procedure

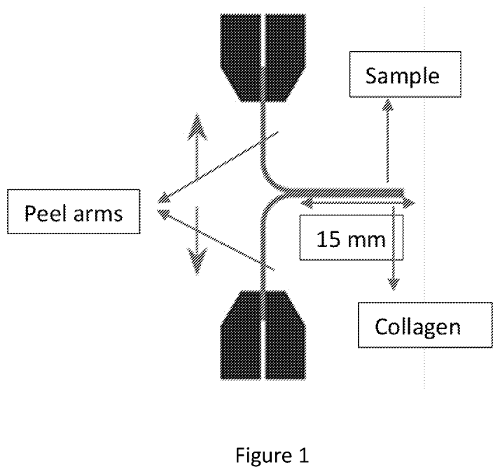

Samples of each matrix layer of the electrospun fibers were cut into 15 mm×30 mm strips; half of the strip was wetted with saline while the other half kept dry. Each strip was placed on top of wet 15 mm×30 mm collagen strip in parallel to create a minimum bonded area of 15×15 mm as can be seen in FIG. 1. The wet half of each sample was pressed against the collagen strip for 2 minutes and the other half was kept unbonded to the collagen to create the peel arms. All samples were left to dry at RT for better handling. The force needed to break the bond between the collagen and the sample was measured using universal testing machine LLOYD LS1. The peel arms are pulled apart at 10 mm/min speed and the force needed to break the bond is measured. The adhesive strength is defined by the average of the peel force and the maximum force recorded during the test.

### Shear Test Procedure

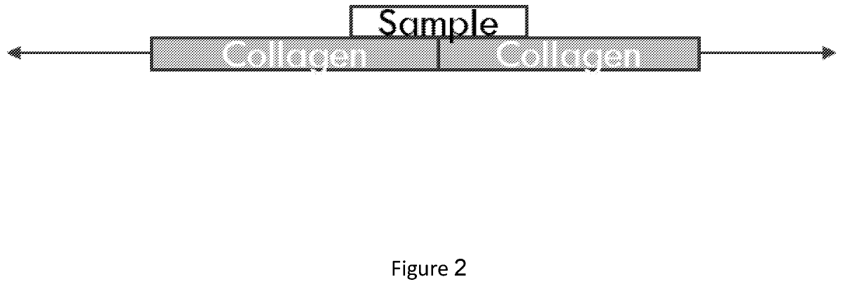

Wet collagen strips were sliced and joined at their slicing points by pressing wet samples against the collagen for 2 minutes as can be seen in FIG. 2. The force needed to break the joint is measured by stretching the collagen strips at 10 mm/min speed using universal testing machine LLOYD LS1. The adhesive strength is defined by the maximum force required to separate the collagen strips at the joint point divided by the bonding area. Commercially available tissue adhesive materials such as Hemopatch™ was also tested to serve as a control.

The adhesion strength determined by the peel test and shear test methods is depicted in FIG. 7 and FIG. 8, respectively. In both tests, Composition 1.2 and Composition 2 (not shown) showed the highest adhesion strength compared to other samples. Consequently, blending of multi arm activated PEG into the electrospinning solution induces an improvement of the adhesion strength, as compared to a single arm activated PEG (except for Composition 1.1 in FIG. 7). Furthermore, according to the results represented by FIG. 7 showing a significant increase of the adhesion strength of composition 1.2 compared to composition 1.1, a combination of the second branched polymer and the third polymer within the fiber of the invention (e.g. Composition 1.2) is preferential over the second branched polymer alone (composition 1.1). Compared to all tested samples, Hemopatch™ appears to have the lowest adhesion strength as determined by the peel test. All the exemplary matrices, according to some embodiments of the invention, having a fibrous structure that keeps its elasticity even after wetting and drying, showed similar results in both tests.

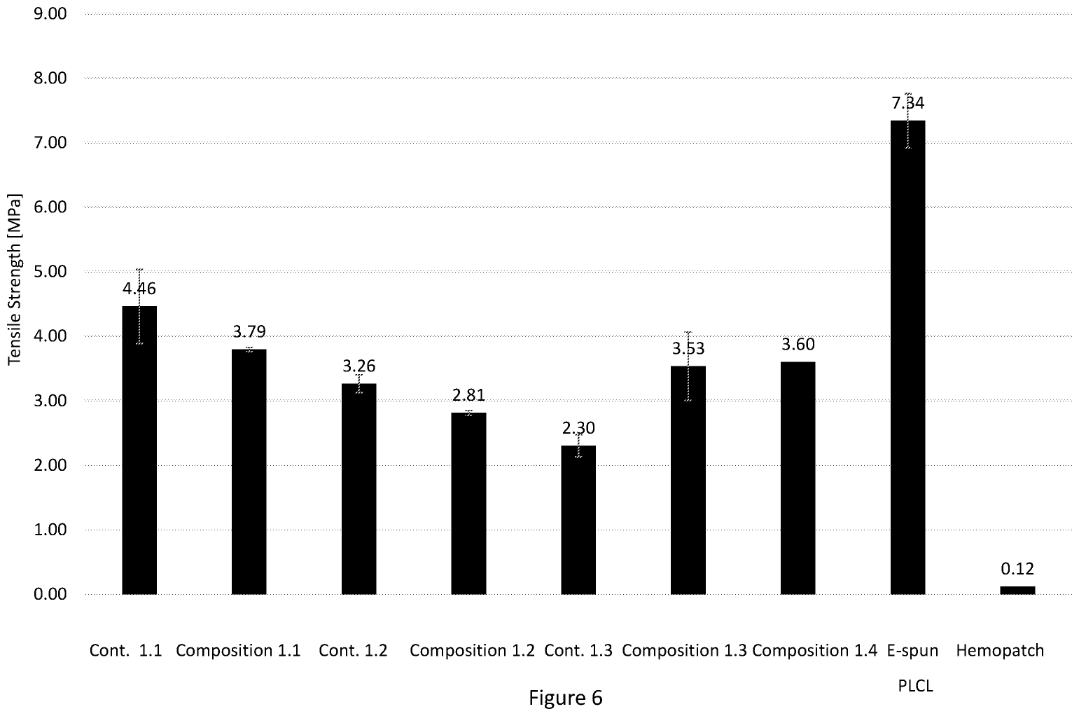

The tensile strength values of the electrospun fibers (Controls 1.1-1.3 and Compositions 1.1-1.4) are presented in FIG. 6. Clear difference can be seen between the samples of blended polymeric fibers and samples of PLCL fibers. The electrospun blended polymeric fibers containing activated PEG appear to fail at lower stresses. This indicates that the mechanical strength of electro-spun PLCL fibers is impaired by the addition of activated PEG. Composition 1.3, however, showed a higher tensile strength compared to its control (Control 1.3). It is speculated, that the blending of m-PEG-NH2 into the electrospinning solution in Composition 1.3, where a higher number of functional groups (NHS) presented, has led to covalent crosslinking between the NHS and NH2 free groups, resulting in stronger fibers and higher tensile strength compared to its control (control 1.3). Composition 1.4 containing multi arm PEG-NH2 and multi arm PEG-NHS, also showed a high tensile strength (3.6 MPa in average) comparable to Composition 1.1 where only multi arm PEG-NHS are presented.

It is noteworthy that the tensile strength of all the tested exemplary compositions of the invention were significantly greater than the tensile strength of DuraGen (a commercial Dural substitute), which ranged between 0.084-0.131 MPa; and greater than the tensile strength of the Hemopatch™ (0.118 MPa).

In summary, the tensile properties such as tensile strength and elongation at break (data not shown) of the tested exemplary compositions of the invention are still significantly higher than the properties of the currently used Collagen products.

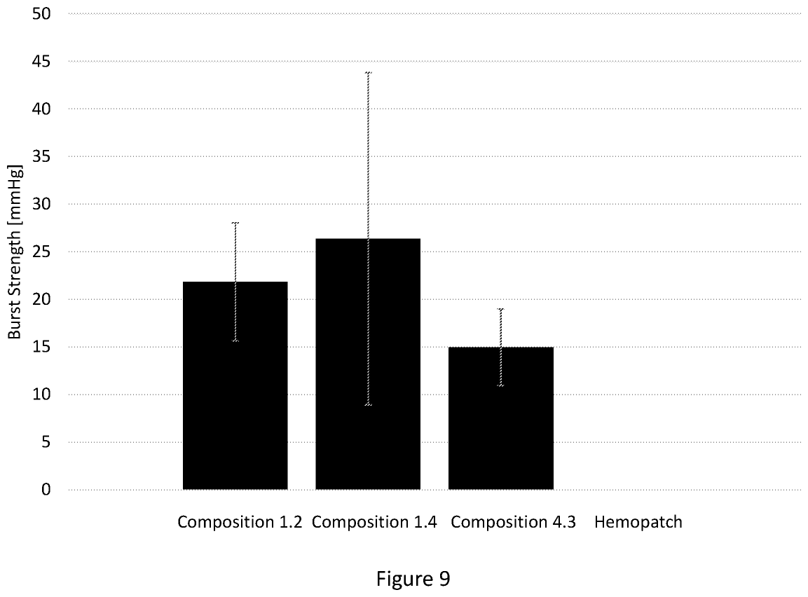

The shear test results represented by FIG. 8 suggest that the tested exemplary compositions of the invention composed of multi arm PEG-NHS containing multi arm PEG-SH has preferred adhesion strength over all prepared prototypes (e.g. Composition 1.2 and Composition 4.3) The addition of multi arm PEG reagent such as PEG-NH2 or PEG-SH increases cohesion strength of the tested compositions (FIG. 9).

Since PEG-NH2 reacts immediately with the NHS groups so that it was almost impossible to obtain an electrospun fiber. In order to overcome this limitation, a kit comprising the electrospun fiber formed by combination of the first polymer (PDCL or PLCL) and of the branched PEG-NHS or of the branched PEG-NH2 was successfully utilized by the inventors. As described hereinabove (Composition 1.4) the tissue reactive component (multi arm PEG-NHS) was applied in situ on top of a fiber layer formed by electrospinning of multi arm PEG-NH2 and PLCL. As can be seen in FIG. 8 and in FIG. 9, the achieved adhesion strength result of Composition 1.4 was within the preferred results.

As shown in FIG. 9, multi arm PEG-SH improves cohesion strength without compromising the adhesion strength of the prepared prototypes in examples 2-4 as can be seen in both adhesion strength tests presented in FIG. 7 and FIG. 8. It can be concluded that the blending of multi arm PEG-SH and PEG-NHS combination into the patch of the PLCL fibers gives the highest adhesion strength (Composition 1.2), the achieved results were also supported by the significant adhesion strength of Composition 1.2 presented in peel test (FIG. 7).

It also can be concluded that the incorporation of the tissue reactive polymers (functionalized PEG reagents) within the PLCL-based fibers induces better adhesion results compared to the coating/distribution methods, this could be due to the high surface-contact area provided by the PLCL-based fibers structure for optimal tissue adhesion. It is worth mentioning that all the achieved results were compared to an available marketed control, Hemopatch™ that is composed of collagen sponge structure, coated with PEG reagents. The averaged adhesion strength of Hemopatch™ was ˜1.15 N. The authors were able to prove that electrospun fiber-based adhesive layer provides enhanced adhesion properties to wet tissue (greater than 1.15N), compared to the currently available commercial products. Furthermore, casted film layers of functionalized PEG polymers blended with PDLC/PLCL (compositions 3.1-4.1) exhibited an impaired adhesion strength compared to the electrospun fiber-based adhesive layers of the invention.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A composition comprising:
(i) a first polymer;
(ii) a second branched polymer;
(iii) a third polymer having reactivity to the second polymer and is at least partially crosslinked to the second branched polymer;
wherein any one of the second and the third polymer comprises a tissue-adhesive group; and wherein (i) said first polymer, and (ii) at least one of said second branched polymer and said third polymer are blended together, so as to form a blended polymeric fiber.

2. The composition of claim 1, wherein said blended polymeric fiber is characterized by a melting point of 50 to 150° C.; wherein said blended polymeric fiber is characterized by an average fiber diameter of 0.5 to 10 um; and wherein an average molecular weight of said first polymer ranges from 10 KDa to 900 KDa.

3. The composition of claim 1, wherein said first polymer is selected from the group consisting of: a polyester, a polyanhydride, a polyacetal, a polyorthoester, a polyurethane, a polycarbonate, a polyphosphazene, a polyphosphoester, a polyether, a silicone, a polyamide, a polysulfone, a polyether ether ketone (PEEK), poly(ethylene glycol), polytetrafluoroethylene, polyethylene, a polysaccharide or any combination or a copolymer thereof.

4. The composition of claim 1, wherein said third polymer is branched and comprises a nucleophilic group.

5. (canceled)

6. The composition of claim 1, wherein said crosslinked is by reacting the tissue-adhesive group and said nucleophilic group.

7. The composition of claim 1, wherein said branched is selected from the group consisting of: a star polymer, a dendrimer, and a hyperbranched polymer or any combination thereof; and wherein said branched comprises three to ten arms.

8. (canceled)

9. The composition of claim 1, wherein said tissue-adhesive group is selected from the group consisting of: an activated ester, an acyl halide, a chloroformate, an anhydride, an aldehyde, an epoxide, an isocyanate, an isothiocyanate, a maleimide, a carbonate, a sulfonyl chloride, a haloacetamide, an acyl azide, an imidoester, a carbodiimide, a vinyl sulfone, ortho-pyridyl-disulfide, or any combination thereof; and wherein said tissue-adhesive group is covalently bound to an arm of said second polymer.

10. (canceled)

11. The composition of claim 1, wherein said second branched polymer, said third polymer, or both are selected from the group consisting of: polyethers, polyesters, polydioxanones, polyphosphoesters, polyurethanes, and polyamides or any combination or a co-polymer thereof.

12. The composition of claim 1, wherein said second branched polymer, said third polymer, or both comprise polyethyleneglycol; and wherein said first polymer is selected from the group consisting of: polylactic acid, poly(L-lactic acid), poly(D-lactic acid), polyglycolic acid, poly(L-glycolic acid), poly(D-glycolic acid), nylon and polycaprolactone or any combination or a co-polymer thereof.

13. The composition of claim 1, wherein an average molecular weight of said second branched polymer, and of said third polymer ranges from 500 Da to 100000 Da.

14. The composition of claim 1, wherein the weight ratio of said third polymer to said second polymer ranges from 1:1 to 1:10; wherein the weight ratio of said first polymer to said second polymer ranges from 1:1 to 20:1.

15. (canceled)

16. (canceled)

17. The composition of claim 1, wherein said blended polymeric fiber is biodegradable.

18. (canceled)

19. (canceled)

20. A matrix, comprising a tissue adhesive layer, wherein said tissue adhesive layer comprises a blended polymeric fiber of claim 1, and wherein said tissue-adhesive layer is characterized by a pore size of between 0.5 to 100 um.

21. The matrix of claim 20, further comprising an additional layer of polymeric fibers, wherein said additional layer enhances a stability of said tissue-adhesive layer.

22. (canceled)

23. (canceled)

24. The matrix of claim 20, wherein said tissue-adhesive layer is characterized by at least one of: a tensile strength of at least 0.05 MPa; an adhesion strength of 1 to 10 N, wherein said adhesion strength is measured according to a shear test; a porosity of at least 60%; a water-permeability of less than 1 ml per hour per cm² upon exposure to an aqueous solution at a pressure of 40 mmHg.

25. (canceled)

26. (canceled)

27. The matrix of claim 20, wherein said tissue-adhesive layer is characterized by a thickness of 0.5 to 250 um.

28. (canceled)

29. The matrix of claim 20, further comprising a pharmaceutically active ingredient.

30. The matrix of claim 20, for use in promoting (i) bioadhesion of at least one biological tissue; (ii) blood coagulation; or for use in repairing and/or substituting a biological tissue.

31. (canceled)

32.-37. (canceled)

38. A process for manufacturing the composition of claim 1, comprising: (i) mixing a first polymer and at least one of the second branched polymer and the third polymer with a solvent, thereby obtaining a solution; and (ii) providing said solution into an electrospinning apparatus.

39. The process of claim 38, wherein said process is for manufacturing a layer of polymeric fibers.
